(19) United States
(12) Patent Application Publication
(10) Pub. No.: US 2015/0265608 A1
(43) Pub. Date: Sep. 24, 2015

(54) Compounds for Treating Rac-GTPase Mediated Disorder

(71) Applicant: Children's Medical Center Corporation, Boston, MA (US)

(72) Inventor: David A. Williams, Dover, MA (US)

(21) Appl. No.: 14/434,916

(22) PCT Filed: Oct. 11, 2013
(86) PCT No.: PCT/US2013/064590

§ 371 Date: Apr. 10, 2015

Related U.S. Application Data
(60) Provisional application No. 61/713,300, filed on Oct. 12, 2012.

(51) Int. Cl. A61K 31/496; A61K 31/4245; A61K 31/4184; A61K 31/42; A61K 31/4162; A61K 31/4439; A61K 31/422

(52) U.S. Cl. CPC A61K 31/496; A61K 31/4439; A61K 31/4245; A61K 31/422; A61K 31/42; A61K 31/4162; A61K 31/4184

(57) ABSTRACT

This disclosure relates to compositions including certain compounds identified by a quantitative, high throughput assay to be effective in the treatment of chronic myelogenous leukemia, as well as methods for the manufacture of and the use of these compounds for treating a Rac-GTPase mediated disorder.

### CROSS-REFERENCE TO RELATED APPLICATION

This application claims priority under 35 U.S.C. §119(e) to U.S. Provisional Application No. 61/713,300 filed Oct. 12, 2012, the disclosure of which is incorporated herein in its entirety.

### BACKGROUND

Rho GTPases comprise a branch of the Ras superfamily of small GTPases. They play a key role in the modulation of a wide array of cellular processes including cell migration, cell polarization, membrane trafficking, cytoskeleton arrangements, proliferation, apoptosis, and transcriptional regulation. (Etienne-Manneville, S. et al (2002). Nature 420, 629-635.; Boettner, B. et al. (2002). Gene 286, 155-174.) Hence, Rho GTPases have been implicated in the pathogenesis of various human diseases including cardiovascular diseases and cancer (Hall, A. Science 1998, 279, 509-514; Wennerberg, K., and Der, C. J. (2004) J. Cell Sci. 117, 1301-1312.; Ridley, A. J. (2006) Trends Cell Biol. 16, 522-529).

The Rho family is comprised of 22 genes encoding at least 25 proteins in humans including Rac. Rho family members bind GTP and transition between an inactive GDP-bound and an active GTP-bound state. In doing so, many of the Rho family members exhibit a GTPase activity when in their active state. This cycling between states is regulated by: guanine nucleotide exchange factors (GEFs); the GTPase activating proteins (GAPs); and GDP dissociation inhibitors (GDIs) which act as negative regulators. (Malumbres, M. et al (2003) Nat. Rev. Cancer 3, 459-465). In quiescent cells, Rho GTPases are predominantly present in an inactive GDP bound state whereas upon growth stimulation, GEFs are activated and subsequently stimulate the guanine nucleotide exchange activity to promote formation of the active GTP bound Rho. When bound to GTP, active Rho GTPases interact with downstream effectors including protein kinases and other proteins with adaptor functions. The intrinsic GTP hydrolysis functionality of Rho GTPases is later stimulated by the Rho specific GTPase activating protein. This returns the Rho protein to its inactive state. Rac-specific RhoGEFs include Tiam1 and Trio (Gao, Y. et al. (2004). Proc. Natl. Acad. Sci. USA 101, 7618-7623.)

The Rac subfamily has also been linked to cellular transformation and hence, the aberrant activity of Rho GTPases is associated with cancer. They play an essential role in transformation caused by Ras and other oncogenes. The Rac1b splice variant of Rac1 has been shown to be constitutively active and transforming; its overexpression has been observed in both breast and colon cancers (Qiu, R. G., et al. (1995) Nature 374, 457-459; Khosravi-Far, R., et al (1995) Mol. Cell. Biol. 15, 6443-6453; Renshaw, M. W. et al (1996) Curr. Biol. 6, 76-83; Ferraro, D., et al. (2006) Oncogene 25, 3689-3698). Rac3 mutants, for example, have been noted in brain tumors and both Rac1 and Rac3 have been linked to glioblastoma invasion (Hwang, S. L. et al (2005) J. Clin. Neurosci. 12, 571-574).

In malignant cells, aberrant Rho GTPase activity results from changes in the expression of Rho GTPases or the perturbed function of either GEFs or GAPs which regulate the function of Rho. (Karnoub, A. E. et al (2004). Breast Cancer Res. Treat. 84, 61-71.) Due to the evidence of Rho involvement in cell transformation, Rho GTPases are probable targets for anti-cancer therapies. Compounds that inhibit GEF interaction with their respective Rho family members would be useful inhibitors of Rho activity and exhibit great specificity. To date, small molecule NSC23766 (i.e., N6-[2-[[4-(diethylamino)-1-methylbutyl]amino]-6-methyl-4-pyrimidinyl]-2-methyl-4,6-quinolinediamine trihydrochloride) has been identified as binding to Rac1 and preventing its activation by Rac-specific RhoGEFs. Some GEF activity, however, was not blocked.

Chronic myelogenous leukemia (CML) is a malignant disease characterized by expression of p210-BCR-ABL, the product of the Philadelphia chromosome. Also known as chronic granulocytic leukemia (CGL), it is a cancer of the white blood cells and is characterized by the increased and upregulated growth of mainly myeloid cells in the bone marrow and the accumulation of these cells in the blood. The deficiency of the Rho GTPases Rac1 and Rac2 in a murine model has shown a significant reduction of p210-BCR-ABL-mediated proliferation. This evidence has strongly suggested Rac as a potential target for leukemia therapy. (E K Thomas et al, Leukemia 22, 898-904, May 2008).

### SUMMARY

This disclosure is based on the discovery of certain anticancer compounds identified through analysis of docking onto the Rac-GTPase protein. In particular, one or more of these compounds identified by this assay unexpectedly exhibited superior activity in inhibiting proliferation of cancer cells with low toxicity to normal cells.

In one aspect, this disclosure features pharmaceutical compositions that include a pharmaceutically acceptable carrier and a compound of formula (I) or a salt thereof (e.g., as an active agent):

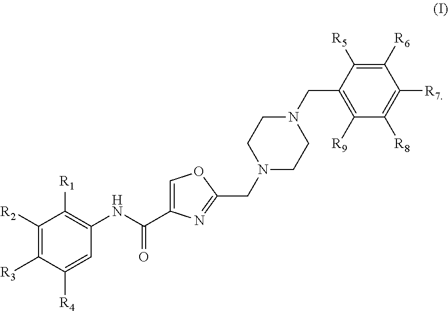

In formula (I), each of R₁, R₂, R₃, R₄, R₅, R₆, R₇, R₈, and R₉, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}, in which each of Rₐ and R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (I), a subset of the compounds described above are those in which R₁, R₂, R₃, R₄, R₅, R₆, R₇, R₈, and R₉, independently, is H, C₁-C₁₀ alkyl (e.g. CH₃), or NRₐR_{b} (e.g., N(CH₃)₂). For example, in these compounds, R₇ can be N(CH₃)₂ and each of R₂ and R₃ can be CH₃. An example of such compounds is

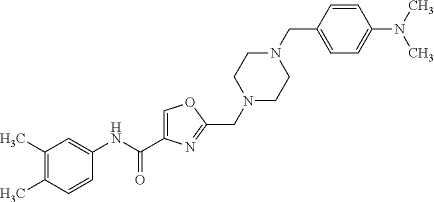

### (i.e., Compound 1).

In another aspect, this disclosure features pharmaceutical compositions that include a pharmaceutically acceptable carrier and a compound of formula (II) or a salt thereof (e.g., as an active agent):

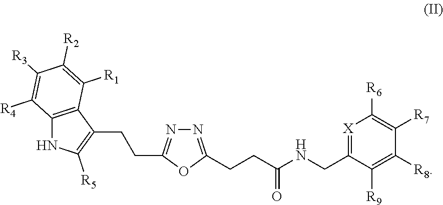

In formula (II), X is N or CH; each of R₁, R₂, R₃, R₄, and R₅, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; each of R₆, R₇, R₈, and R₉, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; or R₆ and R₇, R₇ and R₈, or R₈ and R₉, together with the carbon atoms to which they are attached, are aryl, heteroaryl, C₃-C₂₀ cycloalkyl, or C₁-C₂₀ heterocycloalkyl; each Rₐ, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; and each R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (II), a subset of the compounds described above are those in which X is N. In such compounds, each of R₁, R₂, R₃, R₄, R₅, R₆, R₇, R₈, and R₉, independently, can be H or C₁-C₁₀ alkyl (e.g., CH₂CH₃). For example, in these compounds, R₇ can be CH₂CH₃. An example of such compounds is

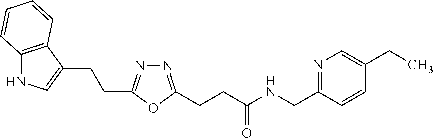 [SMILES: CCc1ccc(CNC(=O)CCc2nnc(CCc3c[nH]c4ccccc34)o2)nc1]

### (i.e., Compound 2).

Referring to formula (II), another subset of the compounds described above are those in which X is CH. In such compounds, each R₆ and R₇, together with the carbon atoms to which they are attached, can be a 1,3-dioxolane group. An example of such compounds is

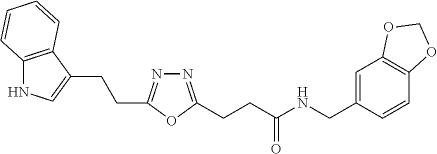 [SMILES: O=C(CCc1nnc(CCc2c[nH]c3ccccc23)o1)NCc1ccc2c(c1)OCO2]

### (i.e., Compound 9).

In another aspect, this disclosure features pharmaceutical compositions that include a pharmaceutically acceptable carrier and a compound of formula (III) or a salt thereof (e.g., as an active agent):

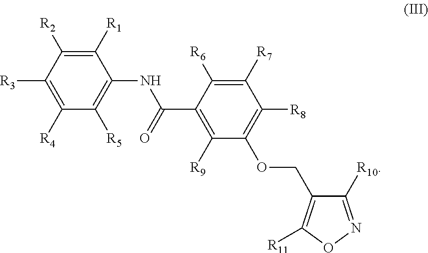

In formula (III), each of R₁, R₂, R₃, R₄, and R₅, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; or R₁ and R₂, R₂ and R₃, R₃ and R₄, or R₄ and R₅, together with the carbon atoms to which they are attached, are aryl, heteroaryl, C₃-C₂₀ cycloalkyl, or C₁-C₂₀ heterocycloalkyl; each of R₆, R₇, R₈, R₉, R₁₀, and R₁₁, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; each Rₐ, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; and each R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (III), a subset of the compounds described above are those in which each of R₃ and R₄ is H, or R₃ and R₄, together with the carbon atoms to which they are attached, are phenyl or a 1,4-dioxane group. In such compounds, each of R₁, R₂, R₅, R₆, R₇, R₈, R₉, R₁₀, and R₁₁, independently, is H, C₁-C₁₀ alkyl (e.g., CH₃), or halo (e.g., Cl). For example, in these compounds, R₁ can be H, Cl, or CH₃; R₂ can be H or Cl; and each of R₁₀ and R₁₁ can be CH₃. Examples of such compounds include

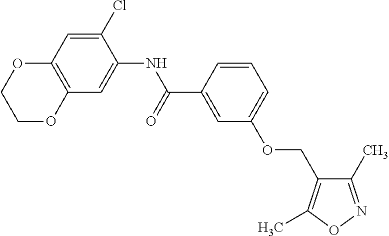 [SMILES: Cc1noc(C)c1COc1cccc(C(=O)Nc2cc3c(cc2Cl)OCCO3)c1]

### (i.e., Compound 3),

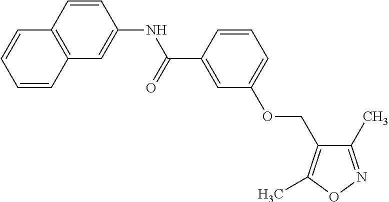 [SMILES: Cc1noc(C)c1COc1cccc(C(=O)Nc2ccc3ccccc3c2)c1]

### (i.e., Compound 6), and

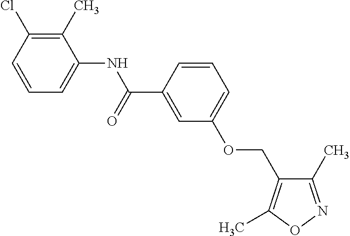 [SMILES: Cc1noc(C)c1COc1cccc(C(=O)Nc2cccc(Cl)c2C)c1]

### (i.e., Compound 8).

In another aspect, this disclosure features pharmaceutical compositions that include a pharmaceutically acceptable carrier and a compound of formula (IV) or a salt thereof (e.g., as an active agent):

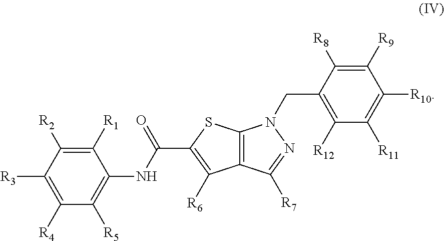

In formula (IV), each of R₁, R₂, R₃, R₄, and R₅, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; or R₁ and R₂, R₂ and R₃, R₃ and R₄, or R₄ and R₅, together with the carbon atoms to which they are attached, are aryl, heteroaryl, C₃-C₂₀ cycloalkyl, or C₁-C₂₀ heterocycloalkyl; each of R₆, R₇, R₈, R₉, R₁₀, R₁₁, and R₁₂, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; each Rₐ, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; each R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (IV), a subset of the compounds described above are those in which each of R₁, R₂, R₆, R₇, R₈, R₉, R₁₀, R₁₁, and R₁₂, independently, is H, C₁-C₁₀ alkyl (e.g., CH₃), or halo (e.g., F). For example, in these compounds, R₁₀ can be F; R₇ can be CH₃; and each of R₃, R₄, and R₅, independently, can be H or S(O)₂N(CH₃)₂; or R₃ and R₄, together with the carbon atoms to which they are attached, can be a 1,3-dioxolane group; or R₄ and R₅, together with the carbon atoms to which they are attached, can be pyrazolyl. Examples of such compounds include

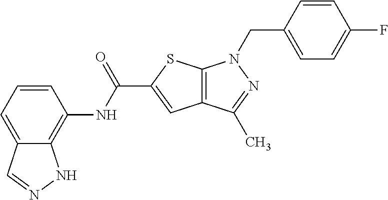 [SMILES: Cc1nn(Cc2ccc(F)cc2)c2sc(C(=O)Nc3cccc4cn[nH]c34)cc12]

(i.e. Compound 4),

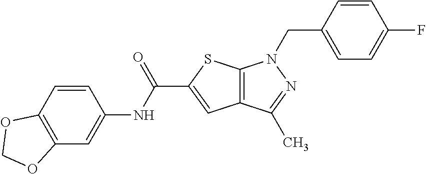 [SMILES: Cc1nn(Cc2ccc(F)cc2)c2sc(C(=O)Nc3ccc4c(c3)OCO4)cc12]

### (i.e., Compound 7), and

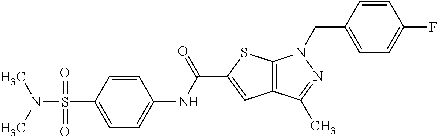 [SMILES: Cc1nn(Cc2ccc(F)cc2)c2sc(C(=O)Nc3ccc(S(=O)(=O)N(C)C)cc3)cc12]

### (i.e., Compound 11).

In another aspect, this disclosure features pharmaceutical compositions that include a pharmaceutically acceptable carrier and a compound of formula (V) or a salt thereof (e.g., as an active agent):

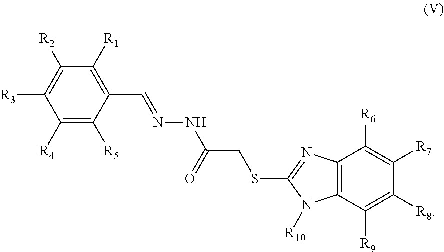

In formula (V), each of R₁, R₂, R₃, R₄, R₅, R₆, R₇, R₈, R₉, and R₁₀, independently, is H, C₁-C₁₀ alkyl optionally substituted with aryl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}, in which each of Rₐ and R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (V), a subset of the compounds described above are those in which each of R₁, R₂, R₃, R₄, R₅, R₆, R₇, R₈, R₉, and R₁₀, independently, is H, OH, Cl, or C₁-C₁₀ alkyl optionally substituted with aryl (e.g., methyl substituted with phenyl). For example, in these compounds, R₁₀ can be methyl substituted with phenyl and each of R₄ and R₅, independently, can be H, Cl, or OH. Example of such compounds include

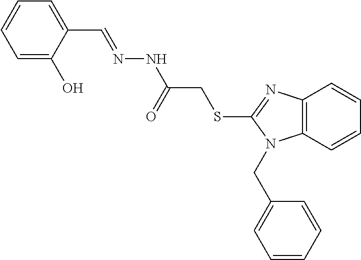 [SMILES: O=C(CSc1nc2ccccc2n1Cc1ccccc1)N/N=C/c1ccccc1O]

(i.e. Compound 5) and

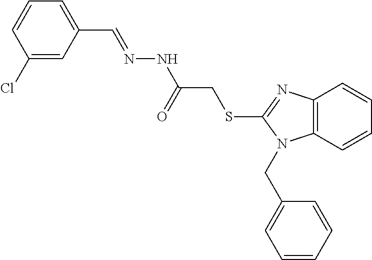 [SMILES: O=C(CSc1nc2ccccc2n1Cc1ccccc1)N/N=C/c1cccc(Cl)c1]

### (i.e., Compound 10).

In another aspect, this closure features pharmaceutical compositions that include a pharmaceutically acceptable carrier and a compound of formula (VI) or a salt thereof (e.g., as an active agent):

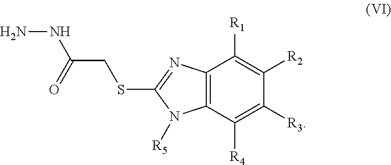

In formula (VI), each of R₁, R₂, R₃, R₄, and R₅, independently, is H, C₁-C₁₀ alkyl optionally substituted with aryl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}, in which each of Rₐ and R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (VI), a subset of the compounds described above are those in which each of R₁, R₂, R₃, R₄, and R₅, independently, is H or C₁-C₁₀ alkyl optionally substituted with aryl (methyl substituted with phenyl). For example, in these compounds, R₅ can be methyl substituted with phenyl. An example of such compounds is

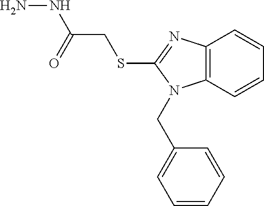 [SMILES: NNC(=O)CSc1nc2ccccc2n1Cc1ccccc1]

### (i.e., Compound 12).

The term “alkyl” refers to a saturated, linear or branched hydrocarbon moiety, such as —CH₃ or —CH(CH₃)₂. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

In still another aspect, this disclosure features a method for treating a Rac-GTPase mediated disorder. The method includes administering to a subject in need thereof an effective amount of one or more of the compounds described above. Examples of Rac-GTPase mediated disorders include cardiovascular diseases, immunodeficiency diseases, inflammatory disorders and cancer. Examples of Rac include Rac1, Rac2, and Rac3. Examples of Rac-GTPase include Rac1-GTPase, Rac2-GTPase, and Rac3-GTPase.

The term “treating” or “treatment” refers to administering one or more of the compounds described above to a subject who has an a disorder treatable with such compounds, and/or a symptom of such a disorder, and/or a predisposition toward such a disorder, with the purpose to confer a therapeutic effect, e.g., to cure, relieve, alter, affect, ameliorate, or prevent the above-described disorder, the symptom of it, or the predisposition toward it.

The compounds described herein include the compounds themselves, as well as their salts, prodrugs, and solvates, if applicable. Examples of prodrugs include esters and other pharmaceutically acceptable derivatives, which, upon administration to a subject, are capable of providing active compounds. A solvate refers to a complex formed between an active compound and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the compounds described above for use in treating an above-described disorder, and the use of such a composition for the manufacture of a medicament for the just-mentioned treatment.

The details of one or more embodiments are set forth in the description below. Other features, objects, and advantages will be apparent from the description, drawings, and claims.

### DESCRIPTION OF DRAWINGS

FIG. 1 depicts the in silico docking of interactions between Rac and its respective GEF, Tiam.

FIG. 2 shows the in silico docking models of the five lead Rac inhibitors which resulted from the initial virtual in silico screen. Compounds 1, 2, 3, 4, and 5 refer to Compounds ALB-H05505197, 47184181, T6590019, PB295833004, and OSSK_{—}373747, respectively.

FIG. 3 is a graph demonstrating the dose dependent inhibition of proliferation in REH cells by 10 lead compounds, which resulted from the initial virtual in silico screen, as measured by MTS assay. The five lead compounds are highlighted in red.

FIG. 4 shows the results of pull down assays for 11 compounds resulting from the virtual in silico screen. The assay indicates disruption of the Rac-GEF interaction. Results for the compound NSC 23766 are also shown. Total Rac1 is shown as a loading control. The 5 lead compounds are highlighted in red.

FIG. 5 is a graph illustrating the dose dependent inhibition of proliferation in SEM cells by 4 of the lead compounds, i.e. Compounds 2, 3, 4, and 5, as measured by MTS assay. Results are shown for the compound concentrations of 5 μM, 10 μM, 20 μM, 40 μM, 80 μM, and 160 μM. Inhibition activity by the compound NSC 23766 is also shown.

FIG. 6 is a graph illustrating the dose dependent inhibition of proliferation in MV411 cells by 4 of the lead compounds, i.e. Compounds 2, 3, 4, and 5, as measured by MTS assay. Efficacy of inhibition of proliferation is shown for each of the compounds at concentrations of 5 μM, 10 μM, 20 μM, 40 μM, 80 μM, and 160 μM.

FIG. 7 is a table highlighting the sensitivity of seven cell lines, i.e. REH, SEM, MV411, RS411, Jurkat, Raji, Nomo-1, Nalm6 and ML2, to 20 μM of the five lead compounds.

FIG. 8 is a graph depicting the average number of colonies per 0.75×10⁵ cells following exposure of normal mouse bone marrow to the lead five compounds independently at 20 or 80 μM. DMSO and ‘no treatment’ are utilized as negative controls. Data following exposure to the compound NSC23766 is also shown.

FIG. 9 collectively indicates the impact of Compound 5, i.e. Compound OSSK_{—}373747, on cellular apoptosis, cell death and GTP-Rac interaction. FIG. 9a is a graph showing the percentage of apoptotic and dead SEM cells following exposure to Compound OSSK_{—}373747. FIG. 9b displays the pull down assay for RAC-GTP in cells exposed to OSSK_{—}373747 for 5 and 60 minutes. The pull down analysis for the compound NSC 23766 is also shown.

FIG. 10 indicates the pharmacokinetics and metabolic stability of Compound OSSK_{—}373747 in vivo.

FIG. 11 illustrates the potential hydrolysis products, product #1 and product #2, of Compound 5, i.e. OSSK_{—}373747.

FIG. 12 lists additional potential lead compounds, i.e. Compounds 6, 7, 8, 9, 10, and 11, identified by the hit expansion drug screen. They are each analogs of the lead compounds, i.e. Compounds 2-5, identified from the initial screen

FIG. 13 displays the inhibition of proliferation of SEM cells by Compounds 6-11 of the hit expansion drug screen at a concentration of 20 and 80 μM.

FIG. 14 displays the inhibition of proliferation of MV411 cells by Compounds 3, 4, 5, 6, 8, 10 and 11 at a concentration of 20 and 80 μM.

FIG. 15 collectively shows the efficacy of the Compounds 6-11. FIG. 15a is a table displaying the sensitivity of seven cell lines, REH, SEM, MV411, Jurjkat, Raji, Nomo-1, and Nalm6, to the new analogs Compounds 6-11. FIG. 15b shows a pull down assay of two analogs, Compounds 6 and 7, after exposure of the compounds to the cells for 5 and 60 minutes. The results for the compound NSC 23766 are also shown. The total Rac protein is shown as a loading control.

FIG. 16 is a graph depicting the percent inhibition of proliferation of SEM cells by 19 compounds attained from the second hit expansion screen. The results for the compound NSC 23766 are also shown. The six selected lead compounds, Compounds 6-11, are highlighted in red.

FIG. 17 is a graph illustrating the dose dependent inhibition of proliferation in Raji cells by one of the analog compounds, Compound 7, i.e., T5602471, as measured by MTS assay. Results are shown for the compound at concentrations of 250 nM, 500 nM, 1 μM, 5 μM, 20 μM, and 80 μM Inhibition activity of the compound NSC 23766 is also shown.

FIG. 18 indicates the pharmacokinetics of Compound 7 in vivo via two routes of administration.

FIG. 19 collectively displays the in vivo tolerability of Compound 7, i.e., T5602471. FIG. 19a is a table of the in vivo tolerability as a measure of plasma concentration of Compound 7 in mice following dosing of 50 mg/kg, 100 mg/kg, 250 mg/kg, and 500 mg/kg. FIG. 19b is a graph displaying the in vivo tolerability of Compound 7 in mice as a measure of body weight following dosing of 50 mg/kg, 100 mg/kg, 250 mg/kg, and 500 mg/kg.

FIG. 20 collectively illustrates the IC₅₀ of Compound 7, i.e., T75602471, following single or double dose of the compound. FIG. 20a is a graph displaying the dose dependent inhibition of proliferation of MV411 cells by Compound 7 as measured by MTS assay. Results are shown for compound concentrations of 250 nM, 500 nM, 1 μM, 5 μM, 10 μM, 20 μM, and 80 μM following a single or a double dose of the compound. Inhibition activity of the compound NSC 23766 is also shown. FIG. 20b is a table highlighting the IC₅₀ of Compound 7 in six leukemia cell lines, i.e. SEM, Nomo, RS411, MV411, Jurkat, and REH, measured following single or double dose of the compound.

### DETAILED DESCRIPTION

This disclosure relates to certain compounds identified as having anti-cancer activity using a quantitative, high throughput assay based on the interactions between the Rho family member Rac and its specific activator GEF, Tiam and the in silico docking of the compounds, individually, on the Rac 2 crystal structure. The compounds unexpectedly exhibit inhibition of leukemia cell proliferation in vitro and, in the case of certain compounds, minimal toxicity to normal bone marrow cells.

All of the compounds described herein can be prepared by methods well known in the art and/or obtained from a commercial source. For example, these compounds can be identified from Evotec AG's EVOsource databases and can be purchased from a commercial source such as Sigma-Aldrich (St. Louis, Mich.). A synthesized compound can be purified by a suitable method such as column chromatography, high-pressure liquid chromatography, or recrystallization.

The compounds described herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

The compounds can be identified by a screening method, such as an assay that identifies compounds that inhibit the proliferation of cancer cells. Alternatively or in addition, compounds can be identified using an assay that identifies compounds that inhibit the activation of the target protein (e.g., Rac-GTPase) and/or by the in silico analysis of the compound docking on the structure of the target protein.

For example, the screening method can include exposing a leukemia cell line, e.g., REM, SEM, MV411, RS411, Jurkat, Raji, Nomo-1, Maim6, and/or ML2, to various doses of the compound for various time periods. A candidate compound that inhibits cell survival can be identified based on the ability of the cell to proliferate in the presence of the compound. Such a screening method can be carried out in a container that includes the cells from a specific cell line, liquid media, and a candidate compound. The container can be, for example, a petri dish, a tissue culture flask, 24-well plate, a 48-well plate, a 96-well plate, a 384-well plate, a 1536-well plate, a 3456-well plate, or any other suitable container. In a high throughput screening method, each well of the container may contain a different candidate compound. As would be appreciated in the art, the screening method may be automated to obtain high throughput. For example, an MTS assay can be performed in liquid medium in standard microtiter plates. In addition, because manual screening of the plates can be slow, labor intensive and subjective, an automated staining method can be used in a high throughput screening method to distinguish live from dead cells.

The present specification also provides pharmaceutical compositions that include at least one (e.g., at least 2, 3, 4, or at least 6) compound(s) depicted in formulae (I)-(VI), (e.g., compounds 1-12), or a pharmaceutically acceptable salt thereof, and a pharmaceutically acceptable carrier.

Compounds described herein can induce inhibition of proliferation. Induction of the inhibition of proliferation can mean inducing or enhancing the suppression of proliferation signals in a cell. For example, induction of the inhibition of proliferation can mean inducing or enhancing cell death in a cell. As another example, induction of the inhibition of proliferation can mean inducing or enhancing apoptosis in a cell. As another example, induction of the inhibition of proliferation can mean inducing or enhancing the state of quiescence in a cell. As yet another example, induction of the inhibition of proliferation can mean inducing or enhancing autophagy. Accordingly, compounds described herein can be used in methods of inducing the suppression of proliferation in a cell, comprising contacting a cell with a compound, salt, or composition described herein, in an amount effective to induce suppression of proliferation in the cell. The contacting can be done in vivo or in vitro.

In some embodiments, this disclosure features a method for treating a Rac-GTPase mediated disorder. The method includes administering to a subject (e.g., a patient) in need thereof an effective amount of a pharmaceutical composition containing one or more of the compounds described above. Examples of Rac-GTPase mediated disorders include cardiovascular disease, immunodeficiency diseases, inflammatory disorders and cancer.

The term “patient” is used throughout the specification to describe an animal, human or non-human, to whom treatment according to the methods of the present invention is provided. Veterinary applications are clearly anticipated by the present invention. The term includes but is not limited to birds, reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs.

Examples of cellular proliferative and/or differentiative disorders include cancer, e.g., carcinoma, sarcoma, metastatic disorders and hematopoietic neoplastic disorders, e.g., leukemias.

A metastatic tumor can arise from a multitude of primary tumor types, including but not limited to those of prostate, colon, lung, breast, bone, and liver origin. Metastases develop, e.g., when tumor cells shed from a primary tumor adhere to vascular endothelium, penetrate into surrounding tissues, and grow to form independent tumors at sites separate from a primary tumor.

The term “cancer” refers to cells having the capacity for autonomous growth. Examples of such cells include cells having an abnormal state or condition characterized by rapidly proliferating cell growth. The term is meant to include cancerous growths, e.g., tumors (e.g., solid tumors); oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

Cancers that may be treated using the methods and compositions of the present invention include, for example, cancers of the stomach, colon, rectum, mouth/pharynx, esophagus, larynx, liver, pancreas, lung, breast, cervix uteri, corpus uteri, ovary, prostate, testis, bladder, skin, bone, kidney, brain/central nervous system, head, neck and throat; Hodgkins disease, non-Hodgkins leukemia, sarcomas, choriocarcinoma, and lymphoma, among others.

Individuals considered at risk for developing cancer may benefit particularly from the invention, primarily because prophylactic treatment can begin before there is any evidence of the disorder. Individuals “at risk” include, e.g., individuals exposed to carcinogens, e.g., by consumption, e.g., by inhalation and/or ingestion, at levels that have been shown statistically to promote cancer in susceptible individuals. Also included are individuals at risk due to exposure to ultraviolet radiation, or their environment, occupation, and/or heredity, as well as those who show signs of a precancerous condition such as polyps. Similarly, individuals in very early stages of cancer or development of metastases (i.e., only one or a few aberrant cells are present in the individual's body or at a particular site in an individual's tissue)) may benefit from such prophylactic treatment.

Other examples of cellular proliferative and/or differentiative disorders that can be treated by the compounds described herein include inflammatory diseases and bone resorption disorders. Examples of inflammatory disorders include neurodegenerative disease, multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, Type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, gout, viral infections, bacterial infections, organ transplant conditions, skin transplant conditions, graft rejection (including allograft rejection and graft-versus-host disease), spondyloarthropathies, scleroderma, vasculitis, and psoriasis (including T-cell mediated psoriasis). Other inflammatory disorders have been described in, e.g., U.S. Application Publication No. 20020155166, the entire contents of which are herein incorporated by reference.

In some embodiments, this disclosure features a method of treating unwanted angiogenesis in a patient. The method includes administering to a patient diagnosed as suffering from or at risk for unwanted angiogenesis an effective amount of a pharmaceutical composition containing one or more of the compounds described herein. The method can optionally include a step of identifying (e.g., diagnosing) the patient as suffering from or at risk for unwanted angiogenesis.

In some embodiments, this disclosure features a method of treating a condition associated with unwanted angiogenesis. The method includes administering to a patient diagnosed as suffering from or at risk for a condition associated with unwanted angiogenesis an effective amount of a pharmaceutical composition containing one or more of the compounds described herein, wherein the condition associated with unwanted angiogenesis is not cancer. The method can optionally include a step of identifying (e.g., diagnosing) the patient as suffering from or at risk for a condition associated with unwanted angiogenesis. In an embodiment, the condition is rheumatoid arthritis, lupus, psoriasis, diabetic retinopathy, retinopathy of prematurity, macular degeneration, corneal graft rejection, neovascular glaucoma, retrolental fibroplasia, rubeosis, Osler-Weber Syndrome, myocardial angiogenesis, plaque neovascularization, telangiectasia, or angiofibroma, or any combination thereof.

### Methods of Treatment

Skilled practitioners will appreciate that a patient can be diagnosed by a physician (or veterinarian, as appropriate for the patient being diagnosed) as suffering from or at risk for a condition described herein, e.g., cancer, by any method known in the art, e.g., by assessing a patient's medical history, performing diagnostic tests, and/or by employing imaging techniques.

Skilled practitioners will also appreciate that compositions described herein need not be administered to a patient by the same individual who diagnosed the patient (or prescribed the composition for the patient). The compositions can be administered (and/or administration can be supervised), e.g., by the diagnosing and/or prescribing individual, and/or any other individual, including the patient her/himself (e.g., where the patient is capable of self-administration).

Amounts of the composition effective to treat a disorder described herein, e.g., cancer, can be administered to (or prescribed for) a patient, e.g., by a physician or veterinarian, on the day the patient is diagnosed as suffering any of these disorders or conditions, or as having any risk factor associated with an increased likelihood that the patient will develop such disorder(s) or condition(s) (e.g., the patient has recently been, is being, or will be exposed to a carcinogen(s)). The composition can be administered to the patient intermittently or continuously. For example, the composition can be administered for at least about 1, 2, 4, 6, 8, 10, 12, 14, 18, or 20 days, or greater than 20 days, e.g., 1 2, 3, 5, or 6 months, or until the patient no longer exhibits symptoms of the condition or disorder, or until the patient is diagnosed as no longer being at risk for the condition or disorder. In a given day, a composition can be administered continuously for the entire day, or intermittently or for up to 23 hours per day, e.g., up to 20, 15, 12, 10, 6, 3, or 2 hours per day, or up to 1 hour per day.

If the patient needs to be treated with chemotherapy, radiation therapy, immunotherapy, gene therapy, and/or surgery (e.g., because prescribed by a physician or veterinarian), the patient can be treated with a composition described herein before, during, and/or after administration of the chemotherapy, radiation therapy, and/or surgery. For example, with regard to chemotherapy, immunotherapy, gene therapy, and radiation therapy, a composition can be administered to the patient, intermittently or continuously, starting 0 to 20 days before the chemotherapy, immunotherapy, gene therapy, or radiation therapy is administered (and where multiple doses are given, before each individual dose), e.g., starting at least about 30 minutes, e.g., about 1, 2, 3, 5, 7, or 10 hours, or about 1, 2, 4, 6, 8, 10, 12, 14, 18, or 20 days, or greater than 20 days, before the administration. Alternatively or in addition, the composition can be administered to the patient concurrent with administration of chemotherapy, immunotherapy, gene therapy, or radiation therapy. Alternatively or in addition, the composition can be administered to the patient after administration of chemotherapy, immunotherapy, gene therapy, or radiation therapy, e.g., starting immediately after administration, and continuing intermittently or continuously for about 1, 2, 3, 5, 7, or 10 hours, or about 1, 2, 5, 8, 10, 20, 30, 50, or 60 days, one year, indefinitely, or until a physician determines that administration of the composition is no longer necessary. With regard to surgical procedures, the composition can be administered systemically or locally to a patient prior to, during, and/or after a surgical procedure is performed. The composition can be administered to the patient intermittently or continuously, for 1 hour, 2, hours, 3 hours, 4 hours, 6, hours, 12 hours, or about 1, 2, 4, 6, 8, 10, 12, 14, 18, or 20 days, or greater than 20 days, before the procedure. It can be administered in the time period immediately prior to the surgery and optionally continue through the procedure, or the administration can cease at least 15 minutes before the surgery begins (e.g., at least 30 minutes, 1 hour, 2 hours 3 hours, 6 hours, or 24 hours before the surgery begins. Alternatively or in addition, the composition can be administered to the patient during the procedure, e.g., by topical administration. Alternatively or in addition, the composition can be administered to the patient after the procedure, e.g., starting immediately after completion of the procedure, and continuing for about 1, 2, 3, 5, 7, or 10 hours, or about 1, 2, 5, 8, 10, 20, 30, 50, or 60 days, 1 year, indefinitely, or until the patient no longer suffers from, or is at risk for, cancer after the completion of the procedure.

Treatments for B-cell chronic lymphocytic leukemia can include administration of combination chemotherapeutic regimens. In many instances, combinations of fludarabine with alkylating agents or with monoclonal antibodies can be used for the treatment of B-CLL. For example, fludarabine can be administered in a combination therapy with alkylating agents such as cyclophosphamide or bendamustine. Fludarabine can also be administered in combination with monoclonal antibodies such as alemtuzumab, rituximab, or ofatumumab. Fludarabine can also be administered for the treatment of B-CLL in combination together with all of the following: an alkylating agent, an anthracycline antibiotic, a vinca alkyloid, and a corticosteroid. For example, fludarbine can be administered together with cyclophosphamide, doxorubicin, vincristine and prednisolone.

Treatments for acute lymphoblastic leukemia (ALL) can include administration of the following: prednisone, vincristine, anthracyclines, L-asparaginase, cyclophosphamide.

Treatments for chronic myelogenous leukemia (CML) can include the administration of imatinib. Treatments for prolymphocytic leukemia can include purine analogues, chlorambucil, and various chemotherapy including: cyclophosphamide, doxorubicin, vincristine, prednisone cyclophosphamide, doxorubicin, vincristine and prednisolone, etoposide, bleomycin VAPEC-B, and Alemtuzumab.

Treatments for the diseases encompassing leukemia can include the following therapeutic agents and combinations of these therapeutic regimens: In many instances, combinations of fludarabine, alkylating agents such as cyclophosphamide or bendamustine, monoclonal antibodies such as alemtuzumab, rituximab, or ofatumumab, an anthracycline antibiotic such as doxirubicin, a vinca alkyloid, anthracyclines, L-asparaginase, cyclophosphamide, imatinib, purine analogues, chlorambucil, cyclophosphamide, doxorubicin, vincristine, prednisone cyclophosphamide, doxorubicin, vincristine and prednisolone, etoposide, bleomycin VAPEC-B, and Alemtuzumab and/or a corticosteroid.

### Combination Therapy

In some embodiments, a compound of the present application, or a pharmaceutically acceptable salt thereof, can be used in combination with another therapeutic agent to treat diseases such as cancer. For example, the additional agent can be a therapeutic agent that is art-recognized as being useful to treat the disease or condition being treated by the compound of the present application as described above. The additional agent also can be an agent that imparts a beneficial attribute to the therapeutic composition (e.g., an agent that affects the viscosity of the composition).

The combination therapy contemplated by the invention includes, for example, administration of one or more compound of the present application, or a pharmaceutically acceptable salt thereof, and additional agent(s) in a single pharmaceutical formulation, as well as administration of a compound of the present application, or a pharmaceutically acceptable salt thereof, and additional agent(s) in separate pharmaceutical formulations. Alternatively or in addition, combination therapy can include administering at least two compounds described herein, or pharmaceutically acceptable salts thereof, in the same or separate pharmaceutical formulations. In other words, co-administration shall mean the administration of at least two agents to a subject so as to provide the beneficial effects of the combination of both agents. For example, the agents may be administered simultaneously or sequentially over a period of time.

The agents set forth herein are for illustrative purposes and not intended to be limiting The combinations, which are part of this invention, can be the compounds of the present application and at least one additional agent selected from the compounds discussed in the summary of the invention. The combination can also include more than one additional agent, e.g., two or three additional agents if the combination is such that the formed composition can perform its intended function.

For example, the methods described herein can be used in combination with the therapies and combination therapies recited above.

### Pharmaceutical Formulations and Dosage Forms

When employed as pharmaceuticals, the compounds of the present application can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Also within the scope of this disclosure are pharmaceutical compositions containing at least one compound described above and a pharmaceutical acceptable carrier. Further, this disclosure covers a method of administering an effective amount of the compounds described herein, e.g., in a pharmaceutical composition, to a patient having cancer, e.g., as described herein. “An effective amount” or “an amount effective” refers to the amount of an active compound that is required to confer a therapeutic effect on the treated patient. Effective doses will vary, as recognized by those skilled in the art, depending on the types of diseases treated, route of administration, excipient usage, and the possibility of co-usage with other therapeutic treatment.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typical doses can range from about 0.01 μg/kg to about 50 mg/kg (e.g., from about 0.1 μg/kg to about 25 mg/kg, from about 1 μg/kg to about 10 mg/kg, from about 10 μg/kg to about 5 mg/kg, or from about 0.1 mg/kg to about 1 mg/kg) of body weight per day. In some embodiments, suitable daily doses can range from about 10 μg/kg to about 100 μg/kg of body weight.

To practice the method described in the present disclosure, a composition having one or more compounds described above can be administered parenterally, orally, nasally, rectally, topically, and/or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in buffered saline or 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as TWEENs or SPANs or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active compounds described above can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound described above. Examples of other carriers include colloidal silicon oxide, magnesium stearate, cellulose, sodium lauryl sulfate, and D&C Yellow #10.

The therapeutic compounds can also be prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Methods of formulating suitable pharmaceutical compositions are known in the art, see, e.g., the books in the series Drugs and the Pharmaceutical Sciences: a Series of Textbooks and Monographs (Dekker, N.Y.).

The compounds described above can be preliminarily screened for their efficacy in treating above-described diseases by the whole-organism screening method described herein and then confirmed by additional animal experiments and clinic trials. Other screening methods will also be apparent to those of ordinary skill in the art.

### Synthesis

Compounds of the present application, including salts thereof, can be prepared using known organic synthesis techniques and can be synthesized according to any of numerous possible synthetic routes, for example, by methods analogous to those of Gerard et al. ACS Comb. Sci. 2011, 13, 365 and as further described in the Examples section.

The reactions for preparing compounds of the present application can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the present application can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present application. Cis and trans geometric isomers of the compounds of the present application are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the present application also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present application can also include all isotopes of atoms occurring in the intermediates or final compounds. Isotopes include those atoms having the same atomic number but different mass numbers. For example, isotopes of hydrogen include tritium and deuterium.

All compounds and pharmaceutically acceptable salts thereof, can be found together with other substances such as water and solvents (e.g. hydrates and solvates) or can be isolated.

In some embodiments, the compounds of the present application, or salts thereof, are substantially isolated. By “substantially isolated” is meant that the compound is at least partially or substantially separated from the environment in which it was formed or detected. Partial separation can include, for example, a composition enriched in the compounds of the present application. Substantial separation can include compositions containing at least about 50%, at least about 60%, at least about 70%, at least about 80%, at least about 90%, at least about 95%, at least about 97%, or at least about 99% by weight of the compounds of the present application, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Kits

The present application also includes pharmaceutical kits useful, for example, in the treatment or prevention of a Rac-GTPase mediated disorder (e.g. cancer), which include one or more containers containing a pharmaceutical composition comprising a therapeutically effective amount of a compound of the present application. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The contents of all publications cited herein (e.g., patents, patent application publications, and articles) are hereby incorporated by reference in their entirety.

### EXAMPLES

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

### Example 1

### In Silico Screen for Rac Inhibitors

### Methods

Rac-GEF Interactions

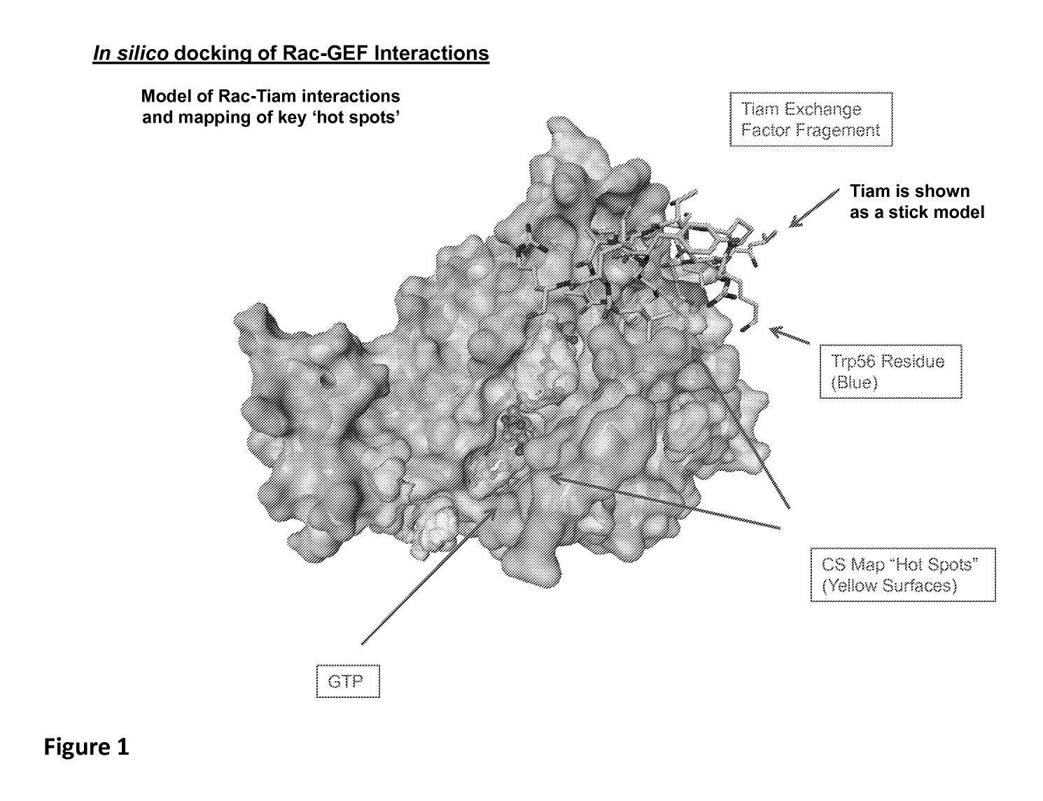

In order to perform a virtual screen for Rac inhibitors, an in silico docking on a Rac2 crystal structure is required. An in silico docking of Rac-GEF interactions was done to decipher potential target sites on the Rac protein on which small molecule binding would result in an interruption in GEF interaction with the protein. FIG. 1 shows a model of the GEF TIAM with RAC (TIAM-Rac). Rac is shown in gray and TIAM is depicted as a stick model. The Trp56 residue of Tiam is highlighted in blue. Mapped are the ‘hot spots’ which are potential locations of interrupting Rac-GEF interactions. The mapped ‘hot spots’, defined as the target locations at which Tiam and Rac have significant interaction, are shown as yellow surfaces. A GTP molecule is also present.

In Silico Screen

A screen of 14 million compounds from the Evotex AG library was performed. Specific filters for drug-like characteristics yielded 4.8 million compounds. These compounds were selected for docking. An in-house algorithm was applied to maximize chemical diversity among the chosen compounds. In silico docking of the compounds on the Rac2 crystal structure and the top 1.2 million compounds were selected for further evaluation. This represented the top 30% of the compounds selected. These compounds were analyzed in two groups. The first group of compounds was analyzed by the docking of the compounds on the Rac1 structure and selected based on a similar binding mode both in Rac-1 and Rac-2, a high docking score both in Rac1 and Rac2 and pharmacophore matching with the binding hypotheses for the known active compounds. Analysis of the second group involved re-scoring the docking poses using ASP and Chemscore scoring functions and selecting compounds with high scoring values for all the 3 scoring methods, i.e. Gold, ASP, Chemsc.

Results

Upon selecting a diverse collection and following visual inspection, 75 compounds were chosen from Group 1. Upon selecting a diverse collection and following visual inspection, 77 compounds were chosen from Group 2. Of these 152 prioritized compounds, 100 of these were purchased from a commercial source for further screening.

### Example 2

### Selection of Lead Compounds

### Methods

Dose Dependent Inhibition of Proliferation

The 100 compounds selected from the initial in silico screen were assayed for inhibition of proliferation in two leukemia cell lines, SEM and REH, by MTS ((3-(4,5-dimethylthiazol-2-yl)-5-(3-carboxymethoxyphenyl)-2-(4-sulfophenyl)-2H-tetrazolium)) assay. The cells for the MTS assay were spun down and re-suspended. The cell suspension was then divided and the compound to be tested added in desired concentrations and subsequently plated. Following an incubation period, MTS reagent (Promega CellTiter 96® Aqueous Non-Radioactive Cell Proliferation Assay) was added and allowed to incubate. The absorbance is subsequently measured at 490 nm using a plate reader.

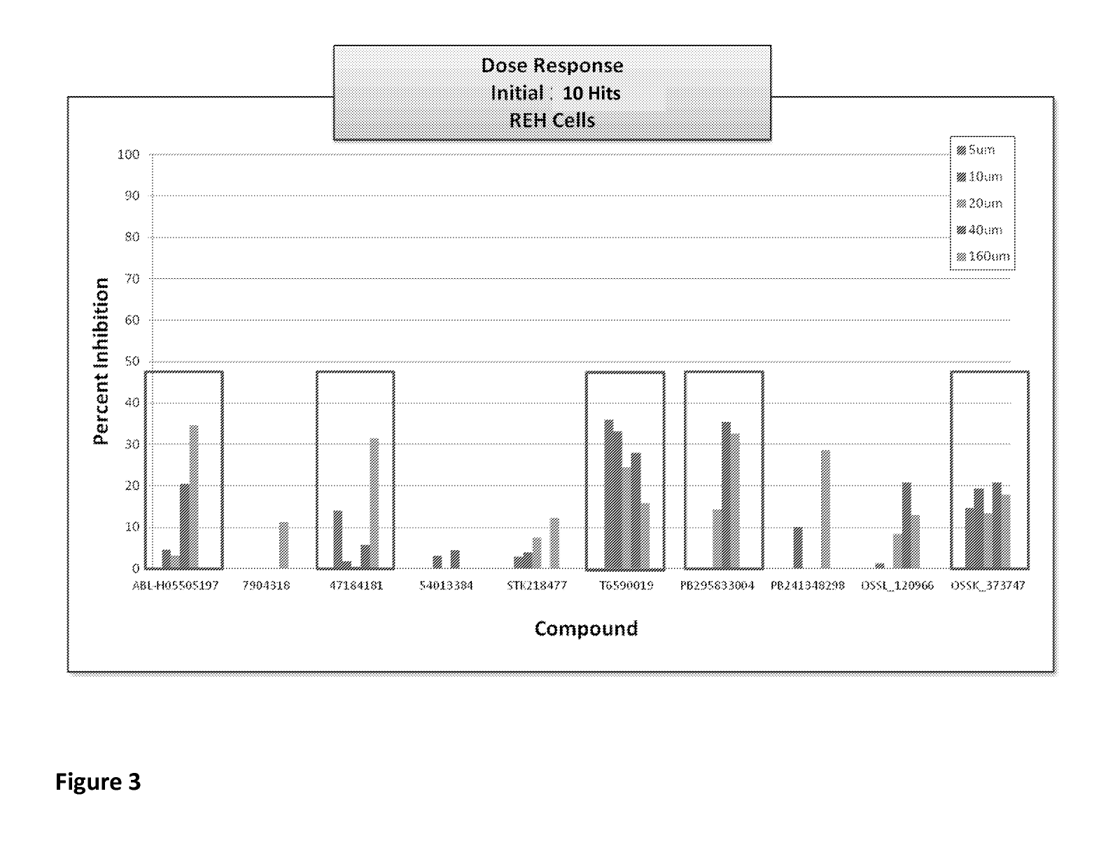

Each compound was analyzed for its effects on cell proliferation. Dose dependent inhibition of proliferation in REH cells by 10 of these compounds individually at 5 μM, 10 μM, 20 μM, 40 μM, and 160 μM, as measured by MTS assay, is shown in FIG. 3.

To determine which of these compounds should be further pursued, a biochemical pull down assay was performed to confirm specific inhibition of Rac activation indicated by disruption of the interaction between Rac and GTPase. The assay initially required treatment of cells followed by pull down and analysis with Western blot. The cells were first starved in serum free media for 2 hours. They were then re-suspended in serum-containing medium and inhibitors added at desired concentration. Following incubation for the desired length of time, the cells were pelleted and lysed with Magnesium Lysis Buffer (Millipore Mg²+lysis/wash buffer). The Rac protein and any bound proteins were then collected with Pak Beads (Millipore Rac/cdc42 Assay Reagent (PAK-1 PBD, agarose). Bound protein was subsequently removed with lysis buffer and subject to Western Blot analysis.

### Results

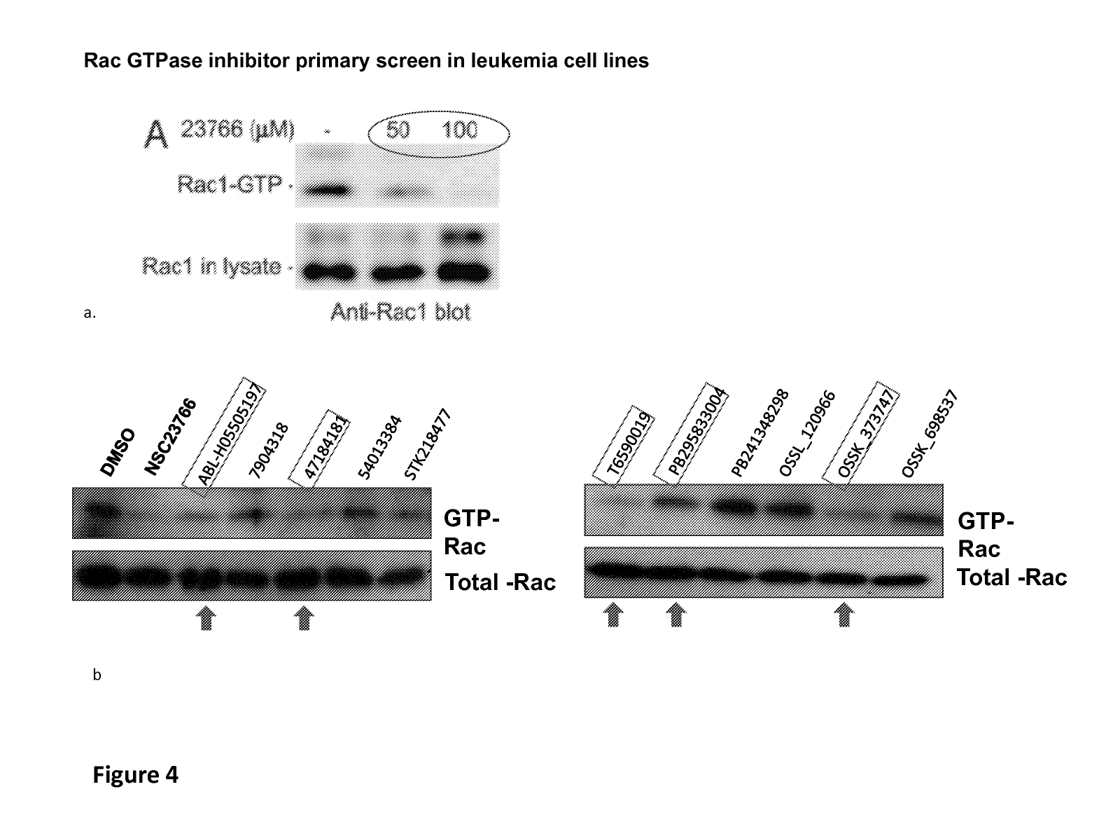

Five compounds (i.e., Compounds 1-5) were identified as lead compounds from this analysis. These compounds are highlighted in red in FIGS. 3 and 4b. A representative Western Blot analysis is shown in which the dose dependent presence of the compound results in the reduction of Rac1-GTPase protein. The Rac1 presence in the lysate is also shown. The pull down analysis is also shown for the compound NSC23776 which is a known small inhibitor of Rac1 binding and of Rac activation by Rac-specific RhoGEFs.

### Example 3

### Analysis of the Lead Compounds (i.e., Compounds 1-5) of Their Efficacy in Inhibiting Leukemia Cell Lines

### Methods

The five lead compounds, i.e. Compounds 1-5, were further analyzed for their effect on cell proliferation at various doses on different leukemia cells. First, each the five compounds was screened in nine leukemia cell lines: REH, SEM, MV411, RS411, Jurkat, Raji, Nomo-1, Naim6, and ML2 at a concentration of 20 μM. Four of the lead compounds, Compounds 2, 3, 4 and 5 were further tested in SEM and MV411 cells at a range of concentrations for inhibition of proliferation. In order to quantitate the percentage inhibition of cell proliferation, MTS analysis was completed as described in Example 2 above.

### Results:

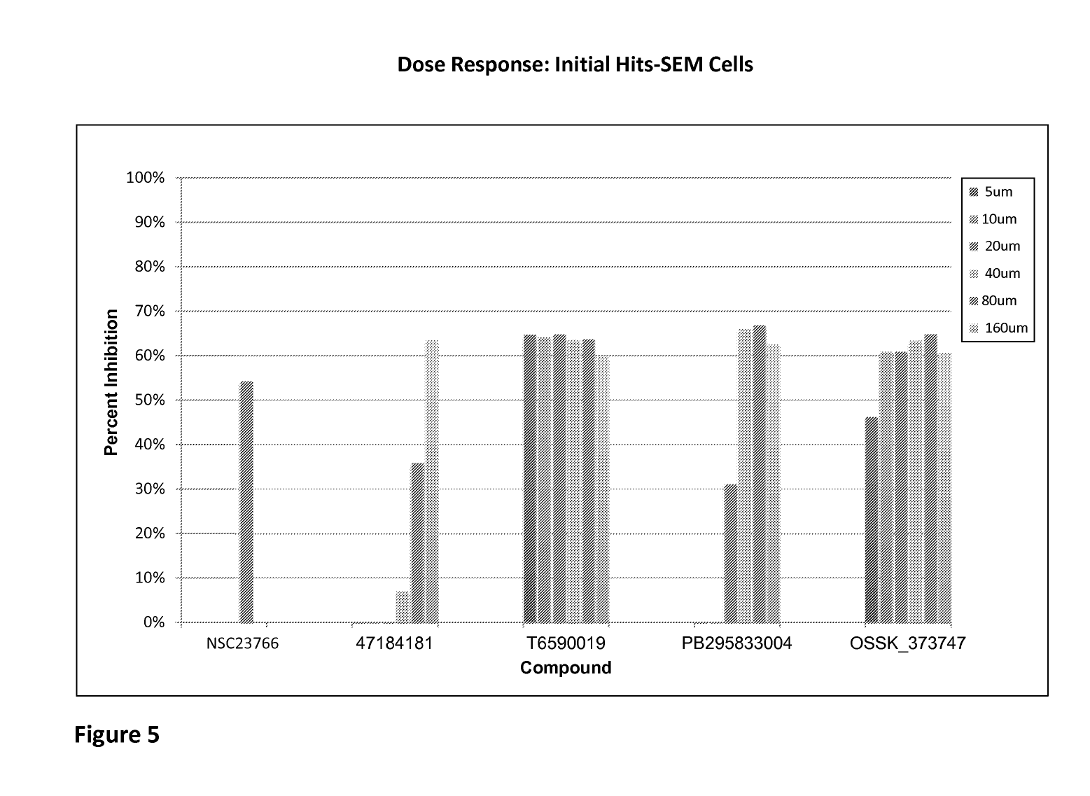

MTS assays were utilized to quantitate the inhibition of proliferation of the cells upon exposure to four of the lead compounds, individually. FIG. 5 shows the percent inhibition of proliferation of SEM cells following exposure to 5 μM, 10 μM, 20 μM, 40 μM, 80 μM and 160 μM of each compound, Compound 2, 3, 4, and 5, individually. Results for the compound NSC23776 are also shown for comparison.

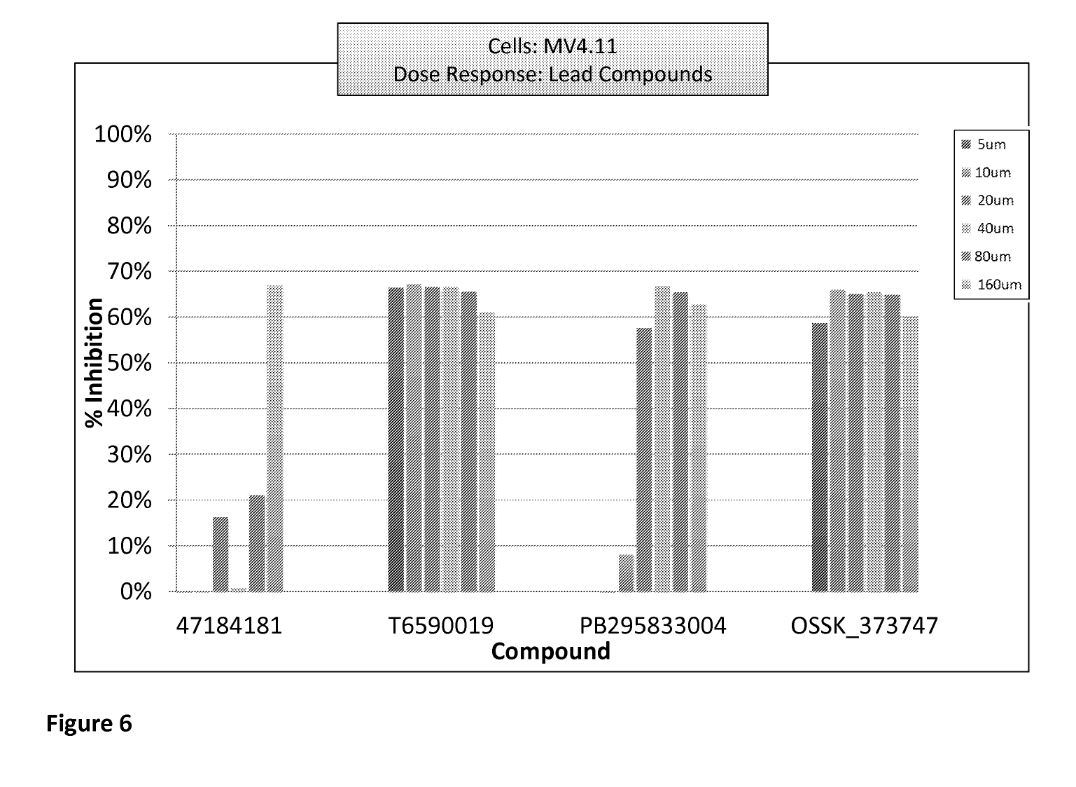

MTS analysis was also performed to ascertain the dose dependent inhibition of proliferation in MV411 cells by four of the lead compounds. Effects on MV411 cells were tested at: 5 μM, 10 μM, 20 μM, 40 μM, 80 μM, and 160 μM of each of Compounds 2, 3, 4, and 5. FIG. 6 depicts the results of the assay performed on MV411 cells in the presence of the compounds at these varying concentrations.

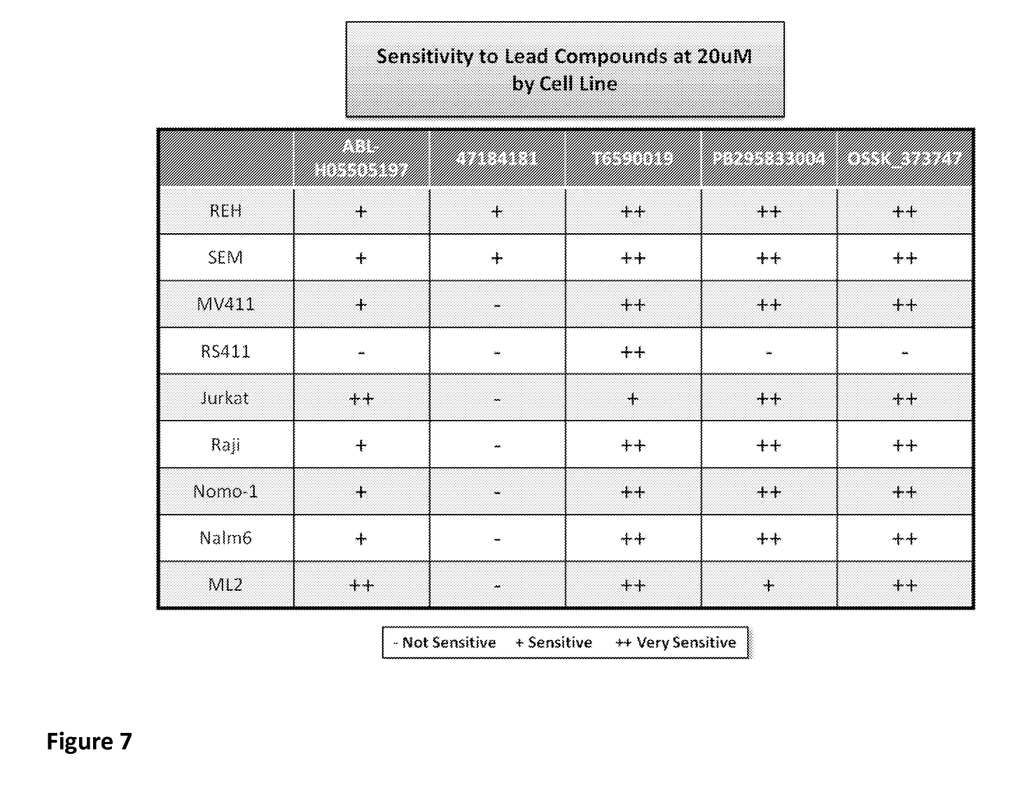

FIG. 7 summarizes the effect of the five lead compounds, Compounds 1-5, on the inhibition of proliferation of nine cell lines at a dose of 20 μM.

### Example 4

### Screening of Compounds 2-5 for Toxicity

### Methods

Four of the lead compounds (Compounds 2, 3, 4, and 5) were screened for toxicity in normal bone marrow hematopoietic progenitor cells. Average colonies formed from normal BL-6 mouse bone marrow were counted following exposure of the four lead compounds individually at concentrations of 5 μM, 20 μM or 80 μM in methylcellulose. A clonogenic assay in methylcellulose was performed to assess colony formation. MethoCult base (StemCell Tech #03134) was mixed with the following reagents: fetal bovine serum, bovine serum albumin, IL-3, mSCF, EPO, beta-mercaptoethanol, penicillin, streptomycin, L-glutamine, and IMDM, and added to normal bone marrow cells. The cells in suspension are then plated and resulting colonies counted after 7 days.

### Results

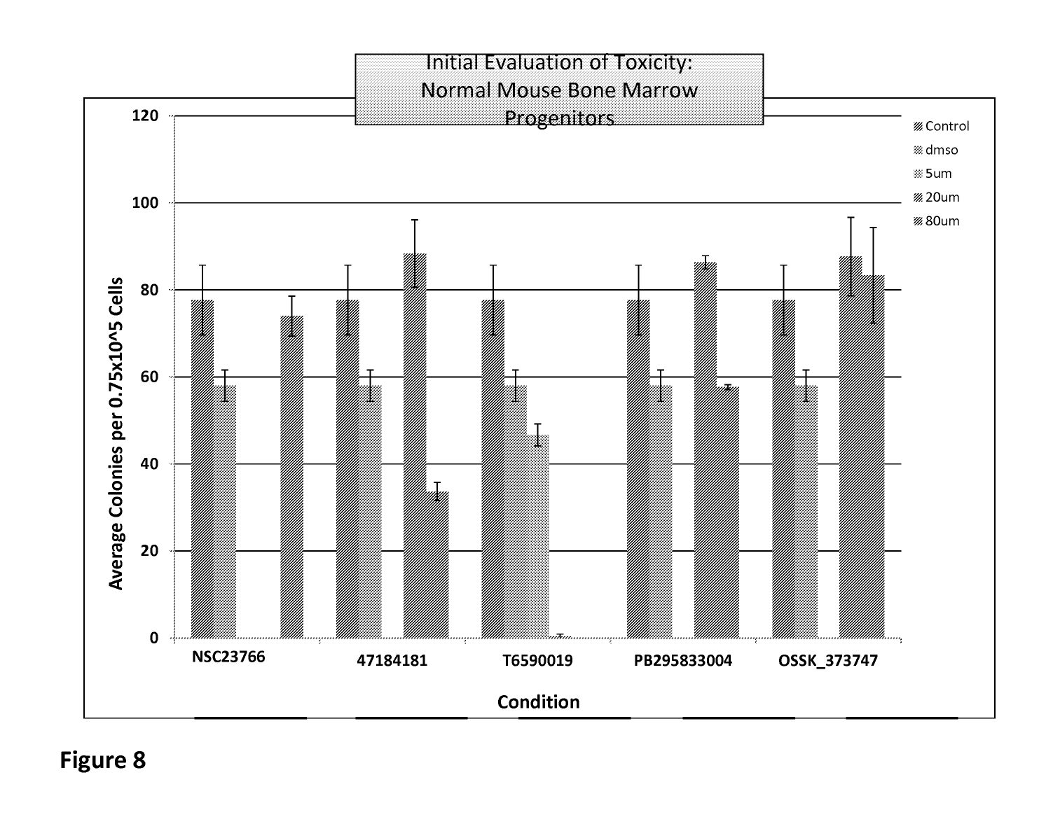

The results of these experiments are graphed in FIG. 8. OSSK_{—}373747 (i.e., Compound 5) demonstrated the least toxicity to the normal cells at either concentration of 20 μM or 80 μM as compared to the other compounds.

### Example 5

### OSSK 373747 Effects on Apoptosis and RAC-GTP Interaction

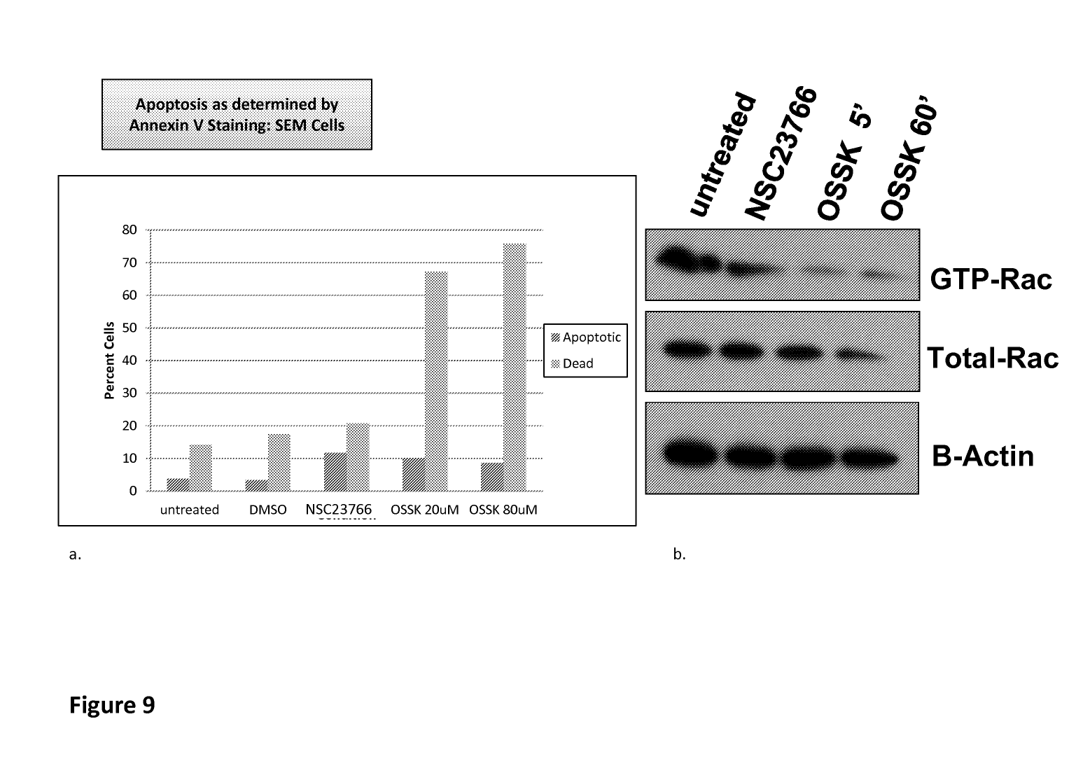

The prior experiments indicated that Compound 5, i.e. OSSK_{—}373747, has strong efficacy but low toxicity as compared to the other compounds. Additional analysis of Compound 5 was performed to assess its effects on apoptosis, cell death and RAC-GTPase interaction. FIG. 9a indicates the effect of OSSK_{—}373747 on the apoptotic pathway in SEM cells. Apoptotic cells and dead cells following incubation of SEM cells with 20 and 80 μM of Compound 5 were analyzed using Annexin V staining. FIG. 9b indicates the decrease in GTPase and Rac interaction in the presence of OSSK_{—}373747 following exposure of the cells for 5 and 60 minutes.

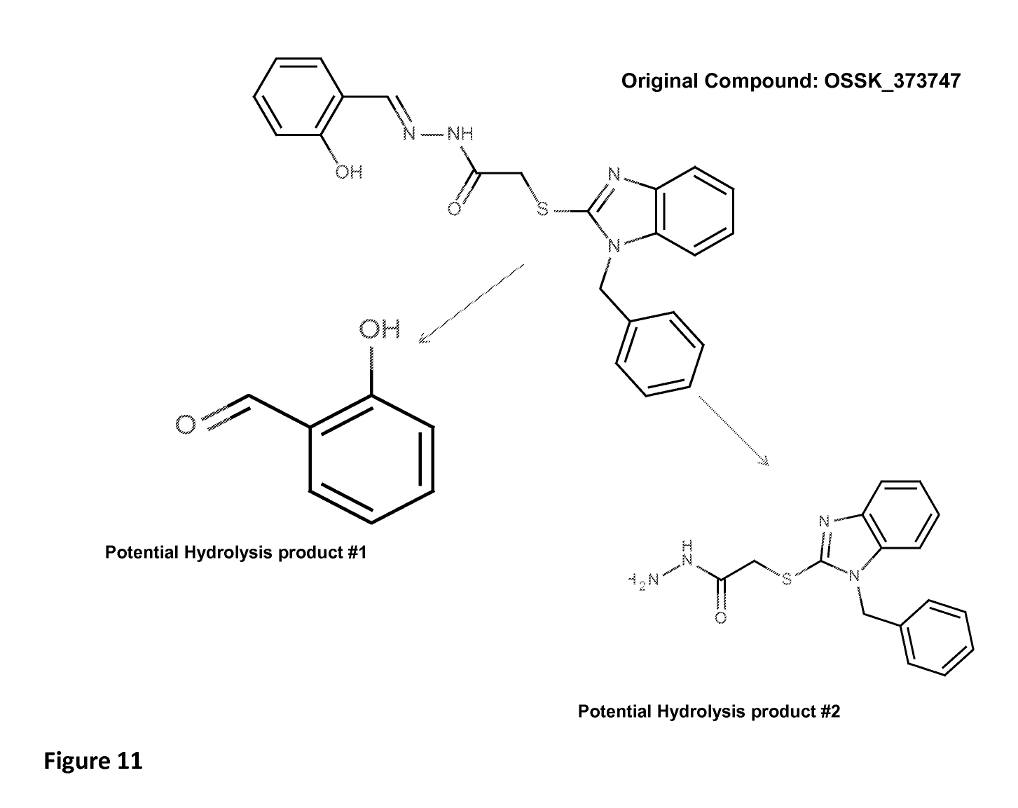

The hydrolysis products for Compound 5 are depicted in FIG. 11. The data has indicated that hydrolysis product #2 (Compound 12) retains inhibitory activity on leukemia cell lines.

### Example 6

### Pharmacokinetics of Compound OSSK 373747 (i.e., Compound 5)

### Methods

Compound 5, i.e., OSSK_{—}373747, was assayed for compound pharmacokinetics in mice. The compound was prepared in a solution containing 10% NMP, 5% Cremphor EL, 30% PEG200, and 55% D5W. Dose levels of 0.5 mg/kg or 1 mg/kg to be given intravenously (IV) and per os (PO), respectively, were administered to male C57BL/6 mice. Prior to compound administration, the animals were fasted for 2 hours and allowed to return to food 4 hours following the dosing.

Serial blood collections were obtained via tail snip. Terminal blood samples were collected via cardiac puncture following inhalation anesthesia and immediately transferred into K₂ETDA tubes on wet ice pending centrifugation (3200 g/10 minutes at 5° C.). Centrifugation was completed within 30 minutes of the collection. Following centrifugation, plasma was separated and transferred into plastic matrix tubes and stored at −80° C. awaiting analytical chemistry. For bone marrow collections, femurs were dissected and bone marrow collected at the specified time points. Samples were stored at −80° C. until analysis.

Blood and bone marrow samples were collected at the following time points: 0.0833, 0.25, 0.5, 1, 2, 3, 4, 6 and 8 hours post administration of the compound either via IV or PO.

### Results

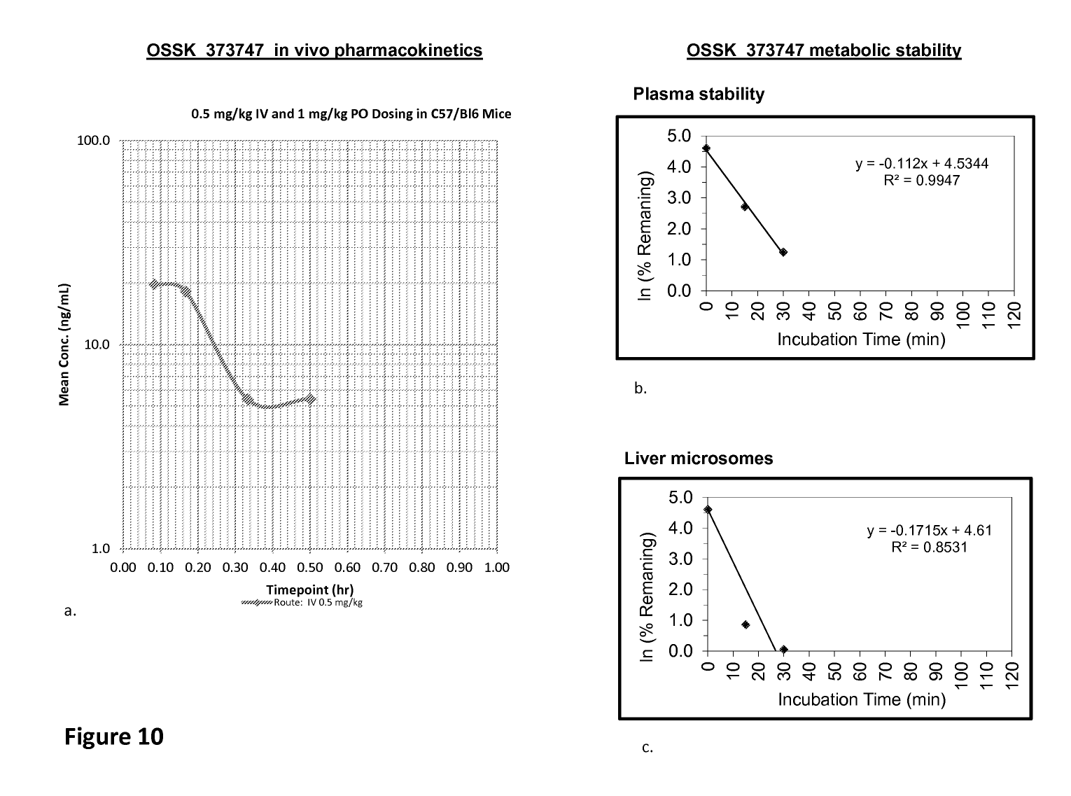

The pharmacokinetics and metabolic stability of Compound 5, i.e., OSSK_{—}373747, is shown in FIG. 10. FIG. 10a is a graph of the pharmacokinetics of the compound following 0.5 mg/kg IV and 1 mg/kg PO in C57/B16 mice. The mean concentration (ng/mL) is indicated over time. FIG. 10b indicates the plasma stability of the Compound OSSK_{—}373747 over time. The percent of compound remaining in the plasma is plotted over time. FIG. 10c indicates the presence of the compound in liver microsomes over time.

### Example 7

### Secondary Screen: Identification of Analogs of the Lead Compounds

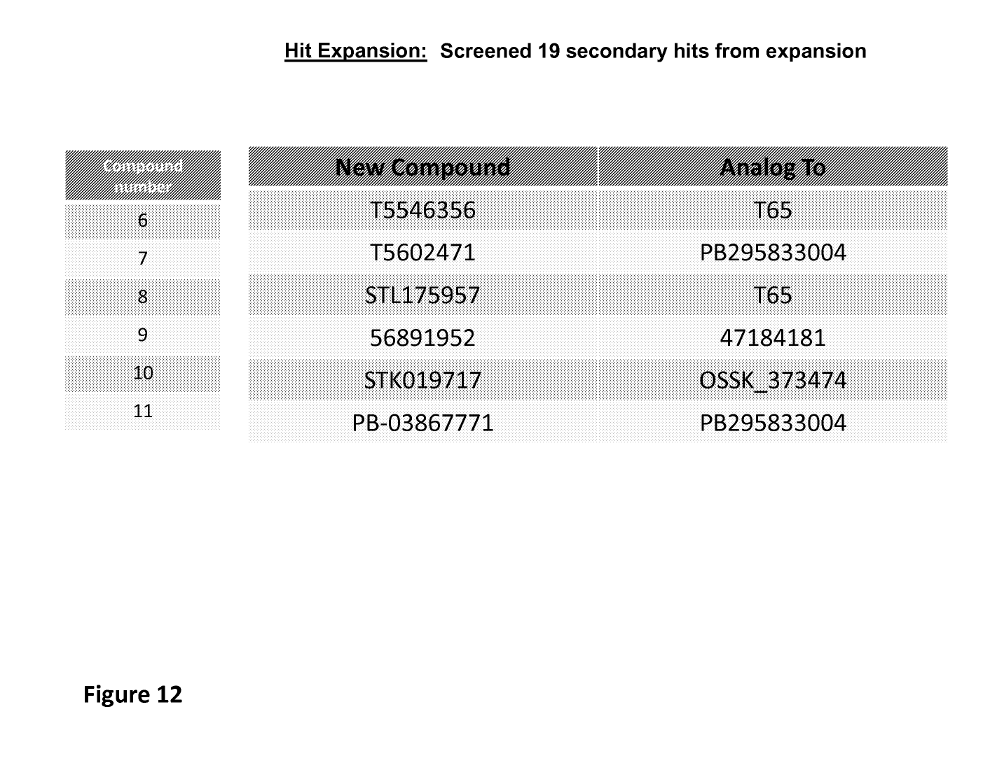

A secondary screen was performed which resulted in the identification of 6 additional compounds (i.e. Compounds 6-11). The table in FIG. 12 lists the new compounds and the lead compounds of which they are analogs.

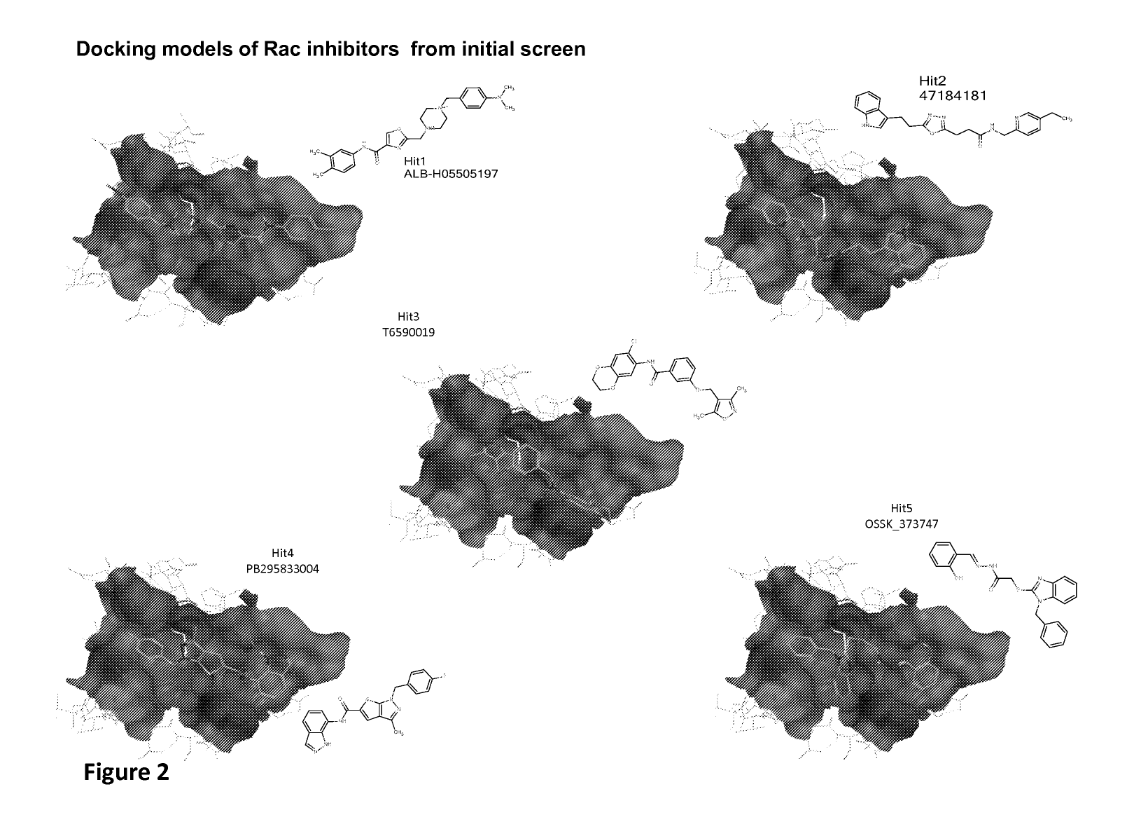

A ‘nearest neighbour’ hit expansion of the 5 lead compounds identified by the initial virtual screening approach, i.e. Compounds 1-5, was performed using close substructure searches and 2D structural similarity of the Evotec EVO source database of about 19.6 million commercially available compounds. The similarity threshold for the similarity searches was set to 0.6 “Tanimoto” similarity with the reference compounds (i.e. Compounds 1-5). About 20,000 compounds were retrieved from the searches and the most similar compounds to the 5 lead compounds were selected. The 20,000 compounds were also subject to an in silico docking experiment on the same Rac protein model used for the initial virtual screen. Compounds which showed convincing docking poses, in agreement with the binding hypotheses used for the virtual screen, were also added to the final list of analogues. FIG. 2 shows the in silico docking of the 5 lead compounds, individually, on the Rac protein.

The selected compounds included ones which were structurally highly similar to the reference hits and which also showed convincing docking hypotheses on RAC. Special care was taken to maximize the variety of compounds selected including performing “intelligent” mutations which were essential to verify the importance of key function of the hits.

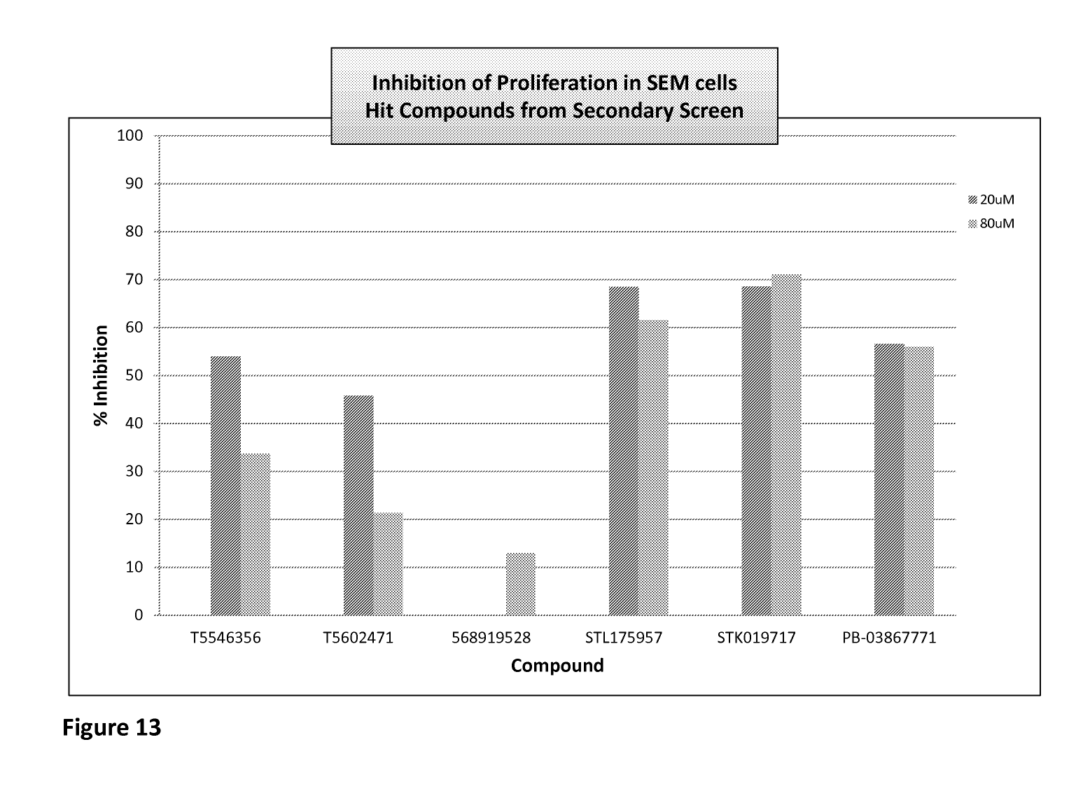

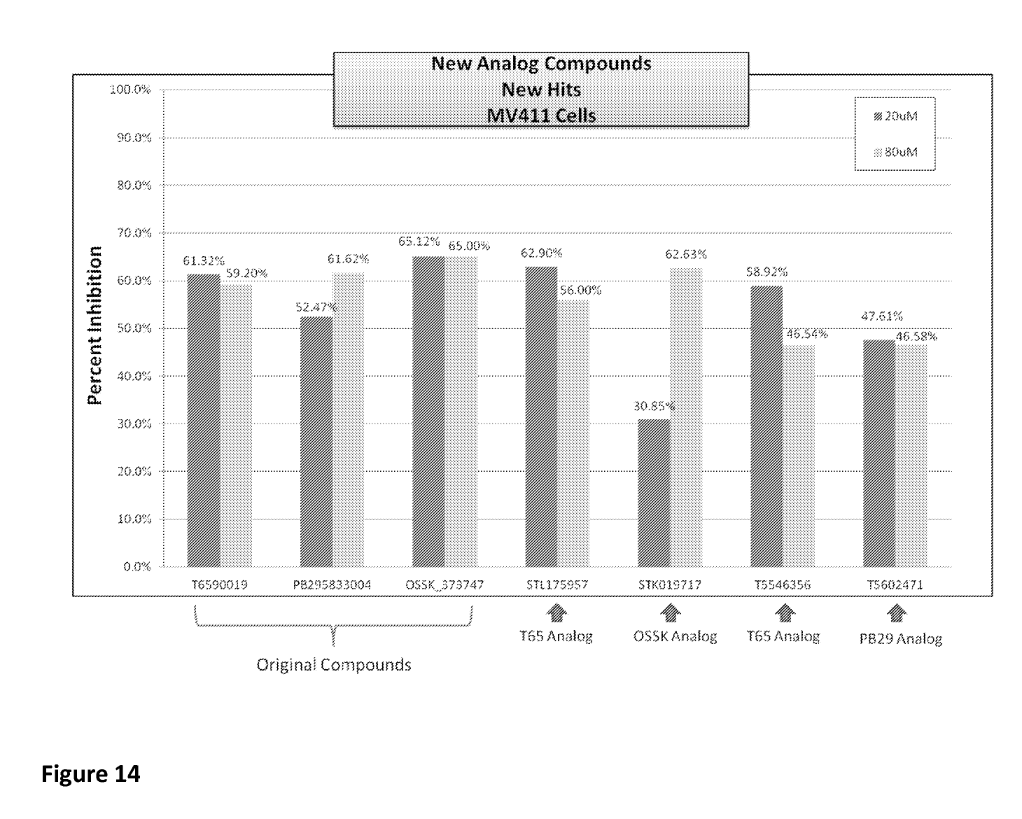

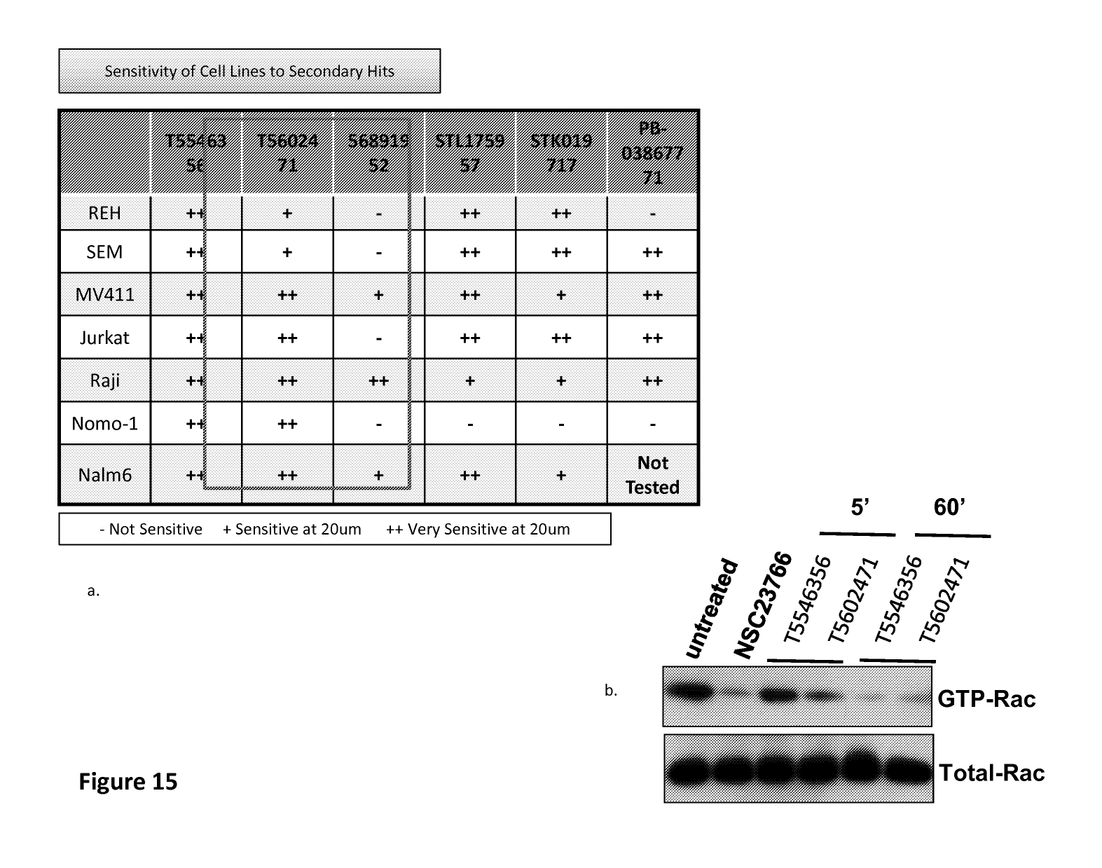

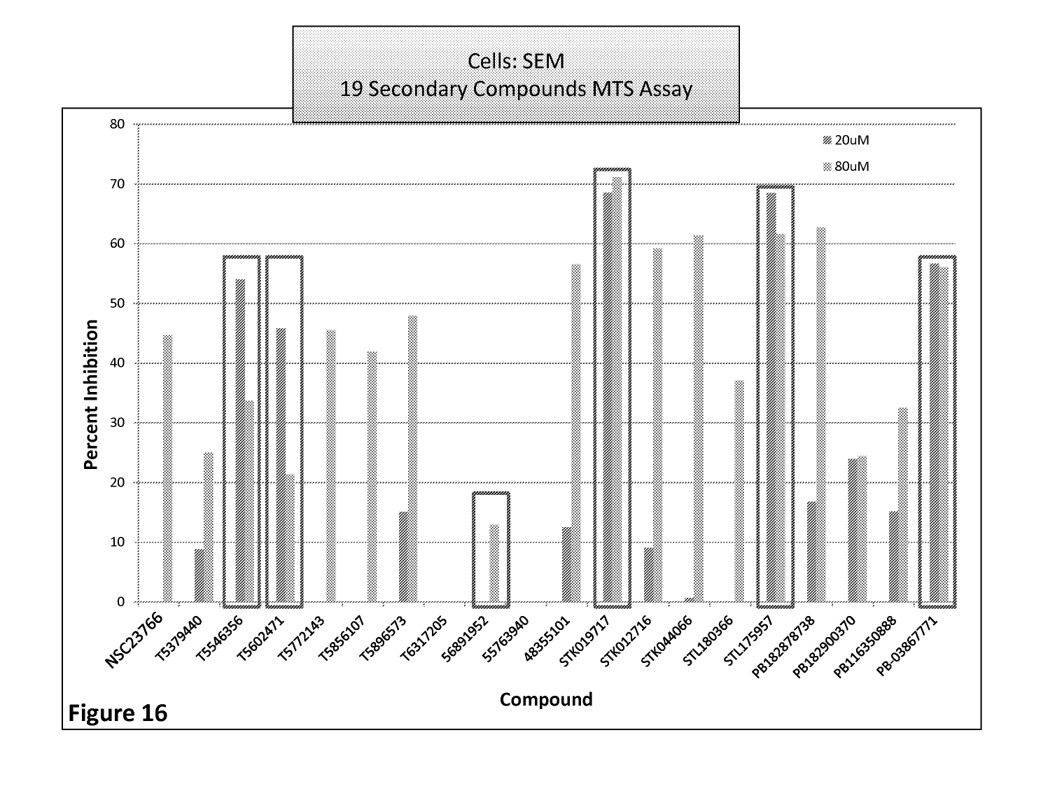

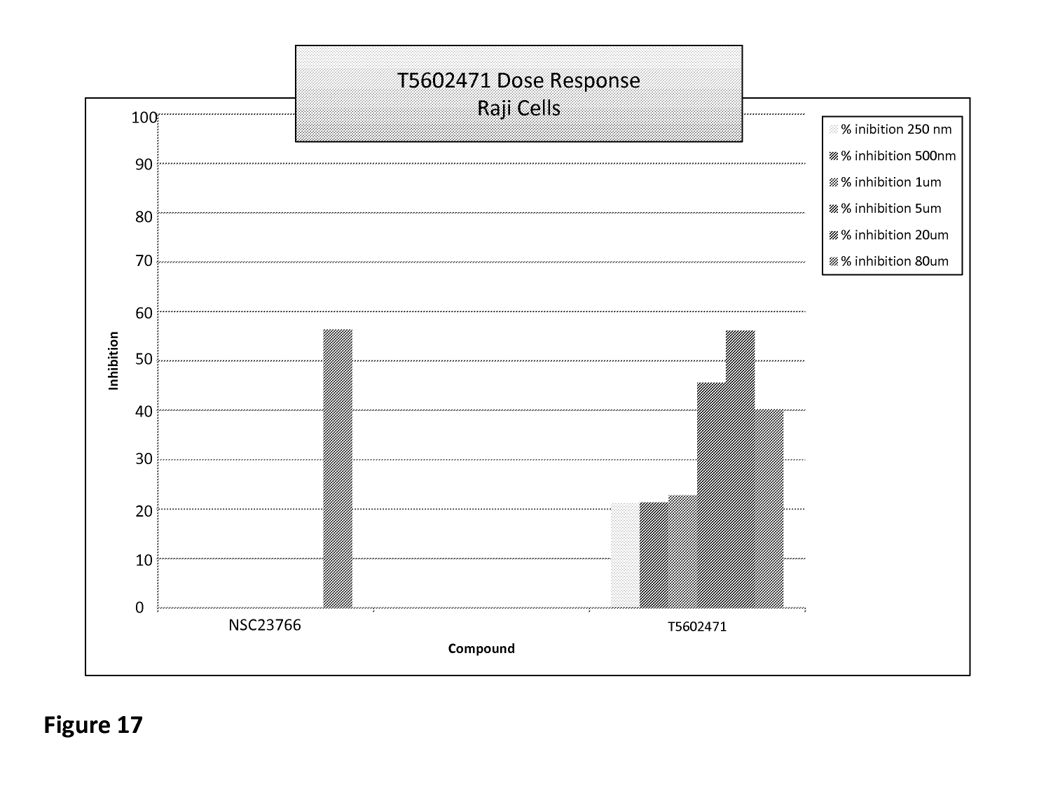

To analyze their efficacy, these 6 analogs were screened for their inhibition of proliferation of various cell lines at both 20 and 80 μM. FIGS. 13 and 14 show the effect of Compounds 6-11 on proliferation inhibition of SEM and Compounds 3, 4, 5, 6, 7, 8, and 10 on proliferation inhibition of MV411 cells, respectively. The table in FIG. 15a shows a comprehensive observation of effects of the analogs on the inhibition of proliferation on seven cell lines: REH, SEM, MV411, Jurkat, Raji, Nomo-1, and Nalm6. A pull down assay was performed on two of the analogs, Compound 6 and Compound 7, following various incubation times. The results of the pull down assay are shown in FIG. 15b. The Rac1 presence in the lysate is also shown. The pull down analysis is also shown for the compound NSC23776 which is a known small inhibitor of Rac1 binding and of Rac activation by Rac-specific RhoGEFs. FIG. 16 indicates the inhibition of proliferation of SEM cells in the presence of 20 and 80 μM of 19 compounds which resulted from the secondary screen. The 6 final lead compounds from this second analysis are highlighted in red.

### Example 8

### Dose Response Analysis of Compound T5602471 (i.e., Compound 7) on Raji Cells

### Methods

Compound 7 was further analyzed for its effect on cell proliferation at various doses on the Raji leukemia cell line. Effect of the compound was assayed in a range of concentrations for inhibition of proliferation. In order to quantitate the percentage inhibition of cell proliferation, MTS analysis was completed as described above in Example 2.

### Results:

MTS assays were utilized to quantitate the inhibition of proliferation of Raji cells upon exposure to Compound 7. FIG. 17 shows the percent inhibition of proliferation of Raji cells following exposure to 250 nM, 500 nM, 1 μM, 5 μM, 20 μM, and 80 μM of Compound 7. Results for the compound NSC23776 are also shown for comparison.

### Example 9

### Pharmacokinetics of Compound T5602471 (i.e., Compound 7)

### Methods

Compound 7, i.e., T5602471, was assayed for compound pharmacokinetics in mice. Dose levels of 0.5 mg/kg or 1 mg/kg to be given intravenously (IV) and per os (PO), respectively, were administered to male C57BL/6 mice. The protocol according to Example 6 was utilized to obtain the pharmacokinetic data for Compound 7.

### Results:

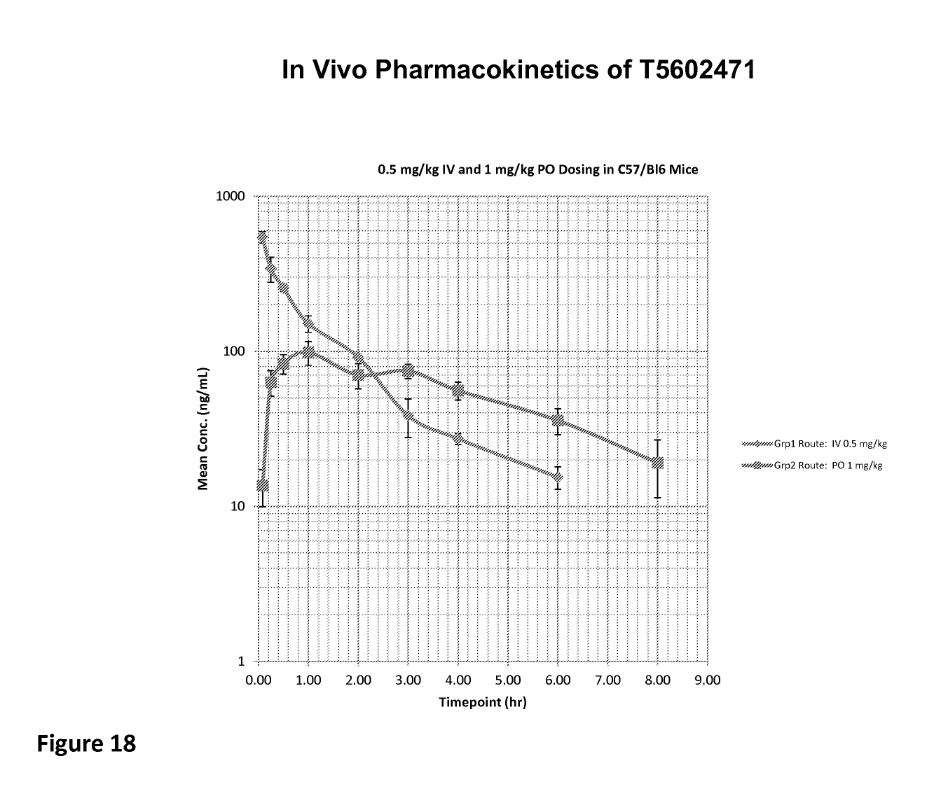

A graph of the pharmacokinetics of Compound 7 following 0.5 mg/kg IV and 1 mg/kg PO in C57/B16 mice is shown in FIG. 18. The mean concentration (ng/mL) is indicated over time for each route of administration.

### Example 10

### In Vivo Tolerability of Compound T5602471 (i.e., Compound 7)

### Methods

In vivo tolerability of Compound 7 was evaluated in NSG mice xenografted with MV411 cells stably expressing luciferase via constitutive promoter. Compound 7 was prepared in a solution of 10% NMP, 5% CremaphorEL, 30% PEG200, and 55% D5W and administered PO at a dose of 50, 100, 250, or 500 mg/kg once each day for a period of 7 days. Five mice were utilized for each test group.

Body weight was recorded 3 times/week upon dosing. On treatment day 6, the mice were imaged via bioluminescence imaging to ensure the presence of the xenografted MV411 leukemia cells. Following the final dose, dose 7, at least 100 μL of blood was drawn via submandibular bleed for plasma collection. Eight hours following the last dose, cardiac puncture blood draw was performed for plasma collection.

### Results:

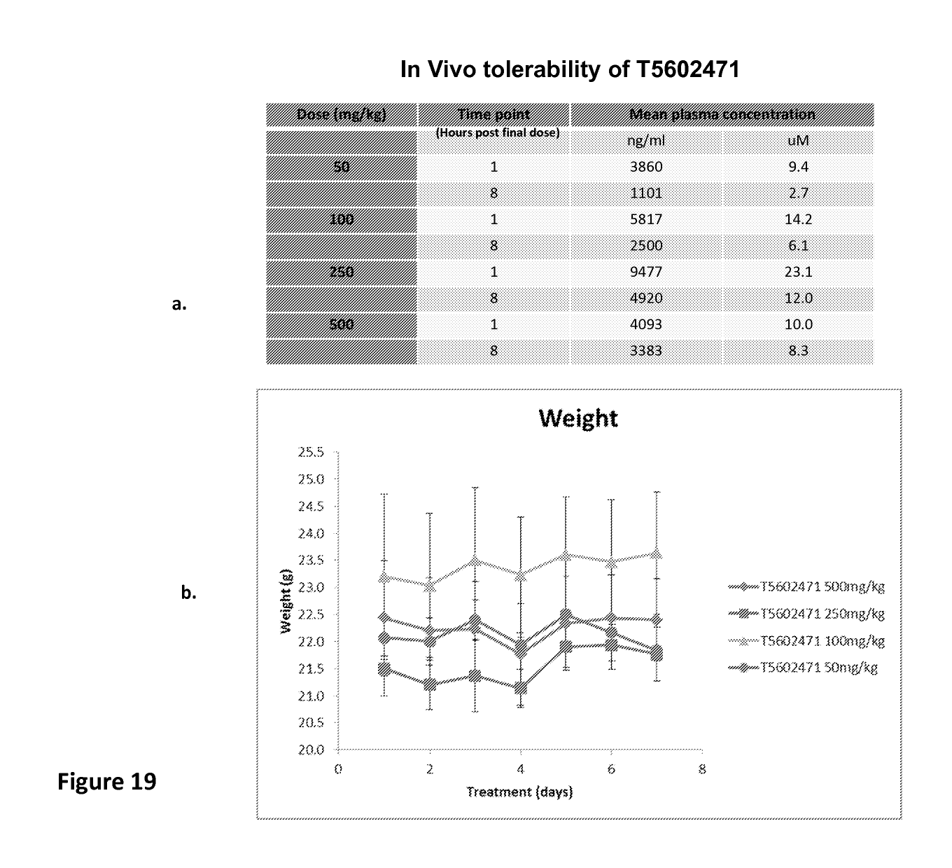

In vivo tolerability of Compound 7 was determined by body weight and plasma concentration in mice following administration of the compound in a range of concentrations: 50, 100, 250, or 500 mg/kg. FIG. 19a shows the mean concentration of Compound 7, i.e., T5602471, in mouse plasma at 1 and 8 hours following the final dose administration of the compound for each concentration tested. The mean plasma concentration is shown in both ng/ml and uM.

FIG. 19b shows the change in weight of animals administered Compound 7, i.e., T5602471, at dose concentrations 50, 100, 250, or 500 mg/kg over a 7 day period. The drug is considered tolerable as there is no progressive weight loss noted at any dose administered.

### Example 11

### IC₅₀ of T5602471 (i.e., Compound 7)

### Methods

Compound 7, i.e., T5602471, was further analyzed for its effect on cell proliferation at various doses on the leukemia cell lines SEM, Nomo, RS411, MV411, Jurket, and REH. Effect of the compound was assayed for its inhibition of proliferation of these cell lines in a range of concentrations. The dosing concentrations of Compound 7 included 250 nM, 500 nM, 1 μM, 5 μM, 20 μM, and 80 μM. The dose response and IC₅₀ was measured at both a single dose and a double dose of Compound 7. For the single dose assay (i.e., 1 addition), Compound 7 was added to the cells at the time of plating. Following a 72 hour incubation, the cells were collected and dose response assay performed. Alternatively, for the double dose assay (i.e., 24 hour additions) Compound 7 was added to the cells at the time of plating, and following a 24 hour incubation additional Compound 7 was added, at the specified concentration, to the cells. Further, following an additional 24 hour incubation (48 hours from the time of plating the cells) additional Compound 7 was added, at the specified concentration, to the cells. After an additional 24 hour incubation (72 hours from the time of plating the cells), the cells were collected and the dose response assay performed.

In order to quantitate the percentage inhibition of cell proliferation, MTS analysis was completed as described above in Example 2.

### Results:

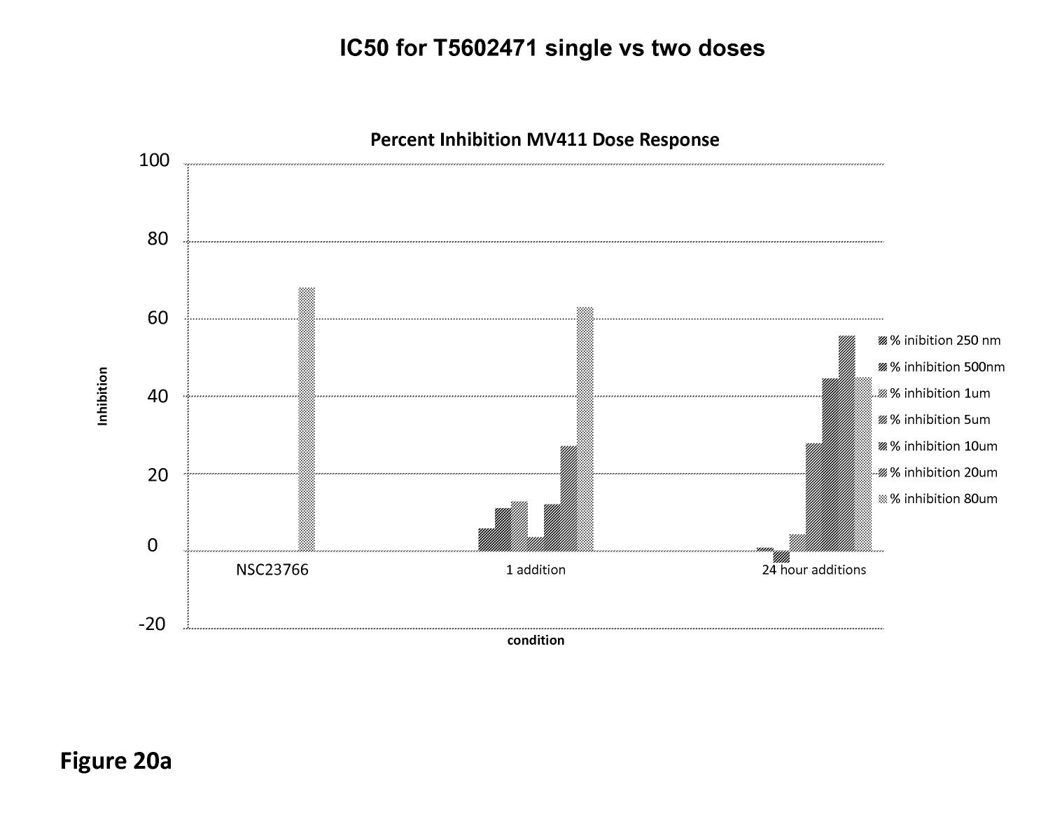

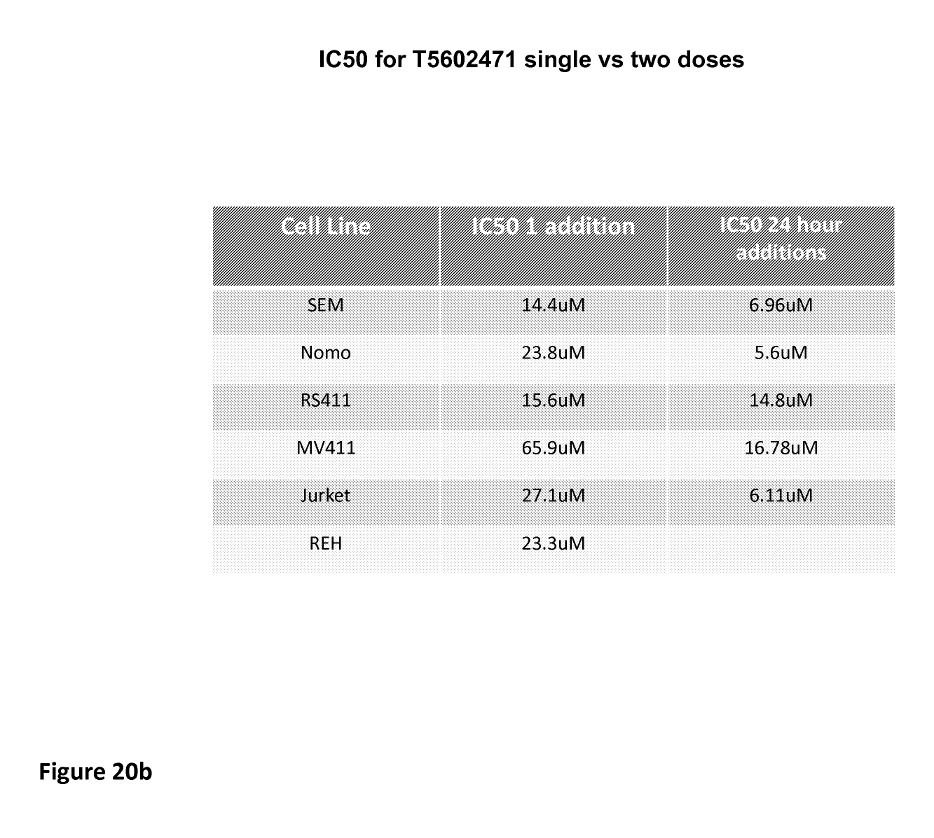

MTS assays were utilized to quantitate the inhibition of proliferation of MV411 cells upon exposure to Compound 7. FIG. 20a shows the percent inhibition of proliferation of MV411 cells following single dose (i.e., 1 addition) and double dose (i.e., 24 hour additions) exposure of 250 nM, 500 nM, 1 μM, 5 μM, 20 μM, and 80 μM of Compound 7. Results for the compound NSC23776 are also shown for comparison. FIG. 20b shows the IC₅₀ of Compound 7 in leukemia cell lines, SEM, Nomo, RS411, MV411, Jurket and REH cells, determined following single dose and double dose administration.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A pharmaceutical composition, comprising a pharmaceutically acceptable carrier and a compound of formula (I) or a salt thereof: 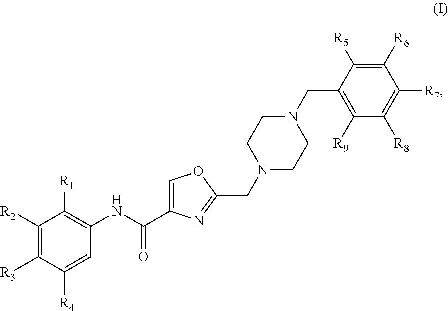
wherein
each of R₁, R₂, R₃, R₄, R₅, R₆, R₇, R₈, and R₉, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}, in which each of Rₐ and R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

2. The composition of claim 1, wherein each of R₁, R₂, R₃, R₄, R₅, R₆, R₇, R₈, and R₉, independently, is H, C₁-C₁₀ alkyl, or NRₐR_{b}.

3. The composition of claim 2, wherein R₇ is N(CH₃)₂.

4. The composition of claim 3, wherein each of R₂ and R₃ is CH₃.

5. The composition of claim 4, wherein the compound is

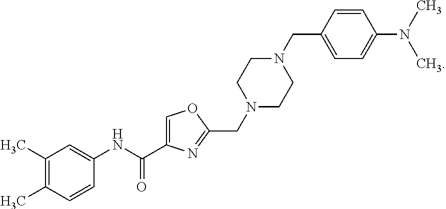

6. A pharmaceutical composition, comprising a pharmaceutically acceptable carrier and a compound of formula (II) or a salt thereof: 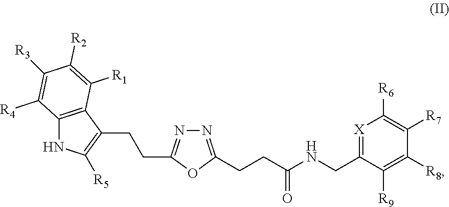
wherein
X is N or CH;
each of R₁, R₂, R₃, R₄, and R₅, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b};
each of R₆, R₇, R₈, and R₉, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; or R₆ and R₇, R₇ and R₈, or R₈ and R₉, together with the carbon atoms to which they are attached, are aryl, heteroaryl, C₃-C₂₀ cycloalkyl, or C₁-C₂₀ heterocycloalkyl;
each Rₐ, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; and
each R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

7. The composition of claim 6, wherein X is N.

8. The composition of claim 7, wherein each of R₁, R₂, R₃, R₄, R₅, R₆, R₇, R₈, and R₉, independently, is H or C₁-C₁₀ alkyl.

9. The composition of claim 8, wherein R₇ is CH₂CH₃.

10. The composition of claim 9, wherein the compound is

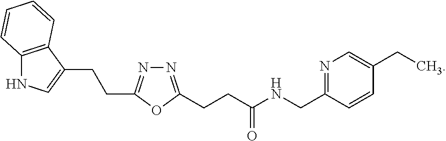 [SMILES: CCc1ccc(CNC(=O)CCc2nnc(CCc3c[nH]c4ccccc34)o2)nc1]

11. The composition of claim 6, wherein X is CH.

12. The composition of claim 11, wherein R₆ and R₇, together with the carbon atoms to which they are attached, are a 1,3-dioxolane group.

13. The composition of claim 12, wherein the compound is

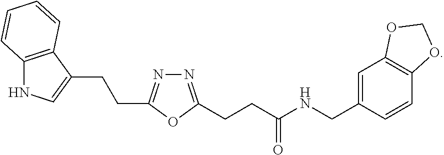 [SMILES: O=C(CCc1nnc(CCc2c[nH]c3ccccc23)o1)NCc1ccc2c(c1)OCO2]

14. A pharmaceutical composition, comprising a pharmaceutically acceptable carrier and a compound of formula (III) or a salt thereof: 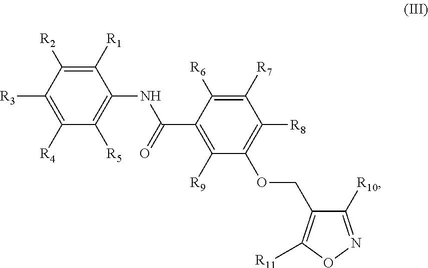
wherein
each of R₁, R₂, R₃, R₄, and R₅, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; or R₁ and R₂, R₂ and R₃, R₃ and R₄, or R₄ and R₅, together with the carbon atoms to which they are attached, are aryl, heteroaryl, C₃-C₂₀ cycloalkyl, or C₁-C₂₀ heterocycloalkyl;
each of R₆, R₇, R₈, R₉, R₁₀, and R₁₁, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b};
each Rₐ, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; and
each R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

15. The composition of claim 14, wherein each of R₃ and R₄ is H; or R₃ and R₄, together with the carbon atoms to which they are attached, are phenyl or a 1,4-dioxane group.

16. The composition of claim 15, wherein each of R₁, R₂, R₅, R₆, R₇, R₈, R₉, R₁₀, and R₁₁, independently, is H, C₁-C₁₀ alkyl, or halo.

17. The composition of claim 16, wherein R₁ is H, Cl, or CH₃; R₂ is H or Cl; and each of R₁₀ and R₁₁, is CH₃.

18. The composition of claim 17, wherein the compound is

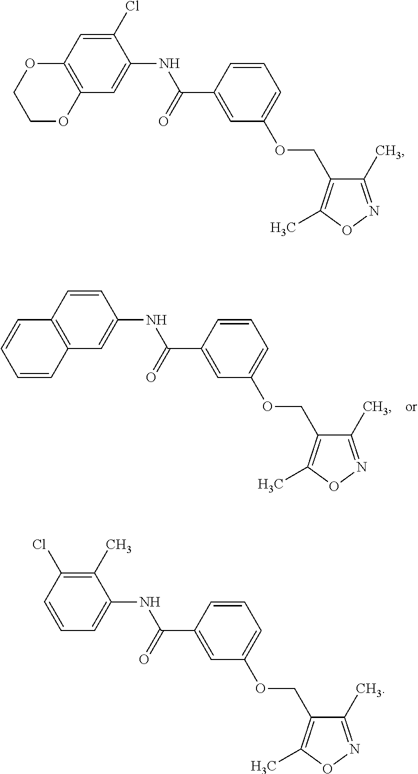 [SMILES: Cc1noc(C)c1COc1cccc(C(=O)Nc2cc3c(cc2Cl)OCCO3)c1 ; Cc1noc(C)c1COc1cccc(C(=O)Nc2ccc3ccccc3c2)c1 ; Cc1noc(C)c1COc1cccc(C(=O)Nc2cccc(Cl)c2C)c1]

19. A pharmaceutical composition, comprising a pharmaceutically acceptable carrier and a compound of formula (IV) or a salt thereof: 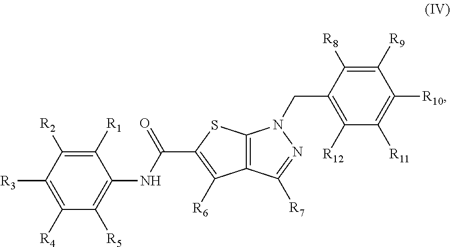
wherein
each of R₁, R₂, R₃, R₄, and R₅, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}; or R₁ and R₂, R₂ and R₃, R₃ and R₄, or R₄ and R₅, together with the carbon atoms to which they are attached, are aryl, heteroaryl, C₃-C₂₀ cycloalkyl, or C₁-C₂₀ heterocycloalkyl;
each of R₆, R₇, R₈, R₉, R₁₀, R₁₁, and R₁₂, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b};
each Rₐ, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl;
each R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

20. The composition of claim 19, wherein each of R₁, R₂, R₆, R₇, R₈, R₉, R₁₀, R₁₁, and R₁₂, independently, is H, C₁-C₁₀ alkyl, or halo.

21. The composition of claim 20, wherein R₁₀ is F.

22. The composition of claim 21, wherein R₇ is CH₃.

23. The composition of claim 22, wherein each of R₃, R₄, and R₅, independently, is H or S(O)₂N(CH₃)₂; or R₃ and R₄, together with the carbon atoms to which they are attached, are a 1,3-dioxolane group; or R₄ and R₅, together with the carbon atoms to which they are attached, are pyrazolyl.

24. The composition of claim 23, wherein the compound is

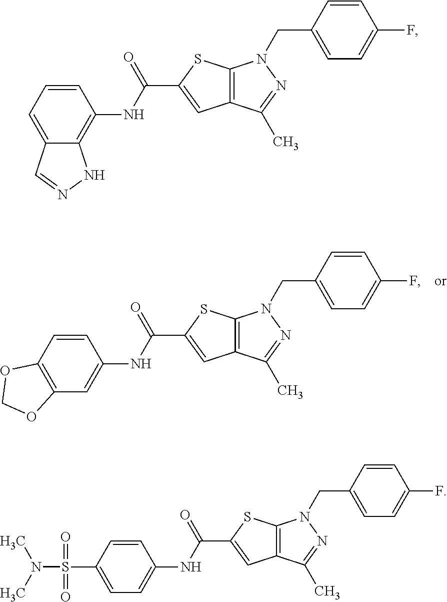 [SMILES: Cc1nn(Cc2ccc(F)cc2)c2sc(C(=O)Nc3cccc4cn[nH]c34)cc12 ; Cc1nn(Cc2ccc(F)cc2)c2sc(C(=O)Nc3ccc4c(c3)OCO4)cc12 ; Cc1nn(Cc2ccc(F)cc2)c2sc(C(=O)Nc3ccc(S(=O)(=O)N(C)C)cc3)cc12]

25. A pharmaceutical composition, comprising a pharmaceutically acceptable carrier and a compound of formula (V) or a salt thereof: 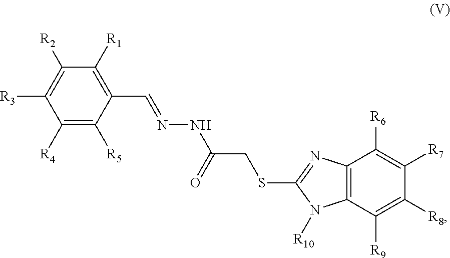
wherein
each of R₁, R₂, R₃, R₄, R₅, R₆, R₇, R₈, R₉, and R₁₀, independently, is H, C₁-C₁₀ alkyl optionally substituted with aryl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}, in which each of Rₐ and R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

26. The composition of claim 25, wherein each of R₁, R₂, R₃, R₄, R₅, R₆, R₇, R₈, R₉, and R₁₀, independently, is H, OH, Cl, or C₁-C₁₀ alkyl optionally substituted with aryl.

27. The composition of claim 26, wherein R₁₀ is methyl substituted with phenyl.

28. The composition of claim 27, wherein each of R₄ and R₅,
independently, is H, Cl, or OH.

29. The composition of claim 28, wherein the compound is

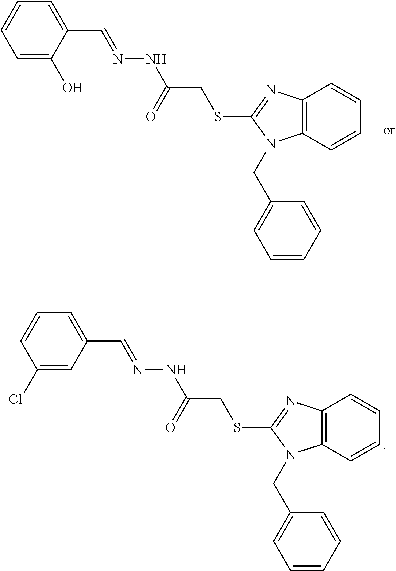 [SMILES: O=C(CSc1nc2ccccc2n1Cc1ccccc1)N/N=C/c1ccccc1O ; O=C(CSc1nc2ccccc2n1Cc1ccccc1)N/N=C/c1cccc(Cl)c1]

30. A pharmaceutical composition, comprising a pharmaceutically acceptable carrier and a compound of formula (VI) or a salt thereof: 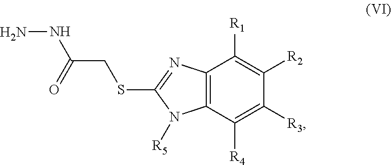
wherein
each of R₁, R₂, R₃, R₄, and R₅, independently, is H, C₁-C₁₀ alkyl optionally substituted with aryl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, halo, ORₐ, SRₐ, COORₐ, OC(O)Rₐ, C(O)Rₐ, C(O)NRₐR_{b}, S(O)₂NRₐR_{b}, or NRₐR_{b}, in which each of Rₐ and R_{b}, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

31. The composition of claim 30, wherein each of R₁, R₂, R₃, R₄, and R₅, independently, is H or C₁-C₁₀ alkyl optionally substituted with aryl.

32. The composition of claim 31, wherein R₅ is methyl substituted with phenyl.

33. The composition of claim 32, wherein the compound is

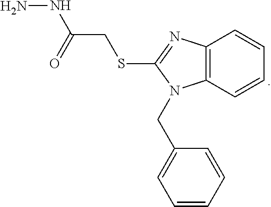 [SMILES: NNC(=O)CSc1nc2ccccc2n1Cc1ccccc1]

34. A method of treating a Rac-GTPase mediated disorder in a subject, comprising administering to the subject in need thereof an effective amount of the pharmaceutical composition of claim 6.

35. The method of claim 34, wherein the Rac-GTPase mediated disorder is cancer.

36. The method of claim 35, wherein the cancer is leukemia.

37. The method of claim 36, wherein cancer is pediatric acute lymphocytic leukemia.

38. The method of claim 34, wherein the Rac-GTPase mediated disorder is an inflammatory disorder.

39. The method of claim 34, wherein the Rac-GTPase mediated disorder is a bone resorption disorder.

40. (canceled)
